SEQUENCE LISTING (88 sequences)

SEQ ID NO: 1 - DNA, 1401 bp, Mus musculus
atgggttggtcctgtatcatcctattcctggtggccactgccacaggtgtgcactccgacgtgcagcttcaggagtcaggacctggcctcgtgaaaccttctcagagtctgtccctcacctgtactgtcactgactactccatcaccagtgattacgcctggaactggatacggcaattcccagggaataaacttgagtggatggggtacataagctacagtggtagcacttcctacaatccatctctcaaaagtcggatctccatcactcgagacacatccaagaaccagttcttcctgcagctgaactctgtgactactgaggacacagccacatattactgtgcatccttcgactatgcccacgccatggattactggggccaagggacttcggtcactgtctcttccgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 2 - protein, 447 aa, Mus musculus
DVQLQESGPGLVKPSQSLSLTCTVTDYSITSDYAWNWIRQFPGNKLEWMGYISYSGSTSYNPSLKSRISITRDTSKNQFFLQLNSVTTEDTATYYCASFDYAHAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 3 - DNA, 702 bp, Mus musculus
atggtatccacacctcagttccttgtatttttgcttttctggattccagcctccagaggtgacatcttgctgactcagtctccagccatcctgtctgtgagtccaggagaaagagtcagtttctcctgcagggccagtcagagcattggcacaagcatacactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattactgtcaacaaattaatagctggccaaccacgttcggcggggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 4 - protein, 214 aa, Mus musculus
DILLTQSPAILSVSPGERVSFSCRASQSIGTSIHWYQQRTNGSPRLLIKYASESISGIPSRFSGSGSGTDFTLSINSVESEDIADYYCQQINSWPTTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - protein, 109 aa, Mus musculus
DILLTQSPAILSVSPGERVSFSCRASQSIGTSIHWYQQRTNGSPRLLIKYASESISGIPSRFSGSGSGTDFTLSINSVESEDIADYYCQQINSWPTTFGGGTKLEIKRA

SEQ ID NO: 6 - protein, 118 aa, Mus musculus
DVQLQESGPGLVKPSQSLSLTCTVTDYSITSDYAWNWIRQFPGNKLEWMGYISYSGSTSYNPSLKSRISITRDTSKNQFFLQLNSVTTEDTATYYCASFDYAHAMDYWGQGTSVTVSS

SEQ ID NO: 7 - protein, 256 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQGHERQSEGSSSPQLQESVFHLLVPSVILVLLAVGGLLFYRWRRRSHQEPQRADSPLEQPEGSPLTQDDRQVELPV

SEQ ID NO: 8 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSSSPQLQESVFHLLVPSVILVLLAVGGLLFYRWRRRSHQEPQRADSPLEQPEGSPLTQDDRQVELPV

SEQ ID NO: 9 - protein, 438 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGHERQSEGSSSPQLQESVFHLLVPSVILVLLAVGGLLFYRWRRRSHQEPQRADSPLEQPEGSPLTQDDRQVELPV

SEQ ID NO: 10 - protein, 441 aa, Mus musculus
EIQLQQSGPELVKTGTSVKISCKASGYSFTGYFMHWVKQSHGKSLEWIGYISCYNGDTNYNQNFKGKATFTVDTSSSTAYMQFNSLTSEDSAVYYCAREGGNYPAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - protein, 214 aa, Mus musculus
DIVMTQSHKFMSTSVGDRVTITCKASQNVGTAVTWYQQKPGQSPKLLIYWTSTRHAGVPDRFTGSGSGTDFTLTISDVQSEDLADYFCQQYSSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 449 aa, Mus musculus
EVKLVESGGGLVQPGGSLKLSCATSGFTFSDYYMYWVRQTPEKRLEWVAYISNGGGSTYYPDTVKGRFTISRDNAKNTLYLQMSRLKSEDTAMYYCARQGSYGYPFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 13 - protein, 214 aa, Mus musculus
AIQMTQTTSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 522 aa, Mus musculus
DVQLQESGPGLVKPSQSLSLTCTVTGYSITSDYAWNWIRQFPGNKLEWMGYISYSGSTSYNPSLKSRISITRDTSKNQFFLQLNSVTTEDTATYYCARLETWLFDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGLDLDDICAEAKDGELDGLWTTITIFISLFLLSVCYSASVTLFKVKWIFSSVVELKQKISPDYRNMIGQGA

SEQ ID NO: 15 - protein, 214 aa, Mus musculus
DILLTQSPAILSVSPGERVSFSCRASQSIGTSIHWYQQRTNGSPRLLIKYASESISGIPSRFSGSGSGTDFTLSINSVESEDIADYYCQQSNSWPTTFGGGTKLEIKWADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 5 aa, Homo sapiens
GYFMH

SEQ ID NO: 17 - protein, 5 aa, Homo sapiens
DYYMY

SEQ ID NO: 18 - protein, 6 aa, Homo sapiens
SDYAWN

SEQ ID NO: 19 - protein, 17 aa, Homo sapiens
YISCYNGDTNYNQNFKG

SEQ ID NO: 20 - protein, 17 aa, Homo sapiens
YISNGGGSTYYPDTVKG

SEQ ID NO: 21 - protein, 16 aa, Homo sapiens
YISYSGSTSYNPSLKS

SEQ ID NO: 22 - protein, 8 aa, Homo sapiens
EGGNYPAY

SEQ ID NO: 23 - protein, 10 aa, Homo sapiens
QGSYGYPFAY

SEQ ID NO: 24 - protein, 9 aa, Homo sapiens
FDYAHAMDY

SEQ ID NO: 25 - protein, 8 aa, Homo sapiens
LETWLFDY

SEQ ID NO: 26 - protein, 7 aa, Homo sapiens
DYGWFDY

SEQ ID NO: 27 - protein, 11 aa, Homo sapiens
KASQNVGTAVT

SEQ ID NO: 28 - protein, 11 aa, Homo sapiens
SASQGISNYLN

SEQ ID NO: 29 - protein, 11 aa, Homo sapiens
RASQSIGTSIH

SEQ ID NO: 30 - protein, 7 aa, Homo sapiens
WTSTRHA

SEQ ID NO: 31 - protein, 7 aa, Homo sapiens
YTSSLHS

SEQ ID NO: 32 - protein, 7 aa, Homo sapiens
YASESIS

SEQ ID NO: 33 - protein, 7 aa, Homo sapiens
YTSESIS

SEQ ID NO: 34 - protein, 9 aa, Homo sapiens
QQYSSYPLT

SEQ ID NO: 35 - protein, 9 aa, Homo sapiens
QQYSKLPWT

SEQ ID NO: 36 - protein, 9 aa, Homo sapiens
QQINSWPTT

SEQ ID NO: 37 - protein, 9 aa, Homo sapiens
QQSNSWPTT

SEQ ID NO: 38 - protein, 9 aa, Homo sapiens
QQYSSWPTT

SEQ ID NO: 39 - protein, 130 aa, Homo sapiens
  misc_feature (23)..(23): Xaa can be any naturally occurring amino acid
DVQLQESGPGLVKPSQTLSLTCXVSGXSXSXXXXXXWIRQPPGKGLEWIGXYYRAXXGXTXYNPSLKSRVTISVDTSKNQFSLXLXSVTAADTAVYYCARXXXXXXXXXXXXXXXXFDXWGQGTXVTVSS

SEQ ID NO: 40 - DNA, 354 bp, Homo sapiens
gacgtacaacttcaagaatctggcccaggtctcgtcaaaccttctcaaactctctcactcacctgcactgttactgactactctattacatccgactacgcttggaactggatccgacaatttcctggtaaaaaactcgaatggatgggttatatttcttactctggctccacctcctacaatccttctctgaaatcacgcatcacaatttcccgcgatacctctaaaaatcaattttcactccaactcaattctgttaccgccgccgatactgccacctactactgtgcctcttttgactacgctcacgccatggattattggggacagggtactaccgttaccgtaagctca

SEQ ID NO: 41 - protein, 118 aa, Homo sapiens
DVQLQESGPGLVKPSQTLSLTCTVTDYSITSDYAWNWIRQFPGKKLEWMGYISYSGSTSYNPSLKSRITISRDTSKNQFSLQLNSVTAADTATYYCASFDYAHAMDYWGQGTTVTVSS

SEQ ID NO: 42 - DNA, 354 bp, Homo sapiens
caagttcaacttcaagaatcaggccccggactcgttaaaccctctcaaactctctctcttacttgcactgtatccgattactctattacttcagactacgcttggaactggatcagacaatttcccggaaaaggactcgaatggatgggatatatctcttactctggctcaacctcttacaacccctctctcaaatctcgaataacaatctcacgcgatacttctaaaaatcaattctcacttcaacttaactccgttactgccgccgacactgccgtttactactgtgcttccttcgattacgcccacgctatggattattggggacaaggaactaccgtcactgtcagctca

SEQ ID NO: 43 - protein, 118 aa, Homo sapiens
QVQLQESGPGLVKPSQTLSLTCTVSDYSITSDYAWNWIRQFPGKGLEWMGYISYSGSTSYNPSLKSRITISRDTSKNQFSLQLNSVTAADTAVYYCASFDYAHAMDYWGQGTTVTVSS

SEQ ID NO: 44 - DNA, 327 bp, Homo sapiens
gaaatagttcttactcaatcccccggtacactctcagtttccccaggcgaacgcgtcactttttcttgcagagcatcacaatcaatcggcacttcaattcattggtatcaacaaaaaacaggacaggccccacgacttcttattaaatatgcatcagaacgagccacaggcatcccagacagattttcaggttcaggatcaggcaccgatttcacacttacaatatccagagtcgaatcagaagattttgcagattactattgtcaacaaataaacagctggcccactacattcggacaaggcacaaaactcgaaattaaacgtacg

SEQ ID NO: 45 - protein, 109 aa, Homo sapiens
EIVLTQSPGTLSVSPGERVTFSCRASQSIGTSIHWYQQKTGQSPRLLIKYASERISGIPDRFSGSGSGTDFTLTISRVESEDFADYYCQQINSWPTTFGQGTKLEIKRT

SEQ ID NO: 46 - DNA, 327 bp, Homo sapiens; identical to SEQ ID NO: 44

SEQ ID NO: 47 - protein, 109 aa, Homo sapiens
EIVLTQSPGTLSVSPGERVTFSCRASQSIGTSIHWYQQKTGQAPRLLIKYASERATGIPDRFSGSGSGTDFTLTISRVESEDFADYYCQQINSWPTTFGQGTKLEIKRT

SEQ ID NO: 48 - protein, 109 aa, Homo sapiens
DILLTQSPAILSVSPGERVSFSCRASQSIGTSIHWYQQRTNGSPRLLIKYASESISGIPDRFSGSGSGTDFTLTISRVESEDFADYYCQQINSWPTTFGQGTKLEIKRT

SEQ ID NO: 49 - protein, 111 aa, Homo sapiens
  misc_feature (98)..(98): Xaa can be any naturally occurring amino acid
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPXTFGQGTKVEIKRT

SEQ ID NO: 50 - protein, 108 aa, Homo sapiens
DVVMTQSPAFLSVTPGEKVTITCQASEGIGNYLYWYQQKPDQAKLLIKYASQSISGVPSRFSGSGSGTDFTFTISSLEAEDAATYYCQQGNKHPLTFGGGTKVEIKRT

SEQ ID NO: 51 - protein, 109 aa, Artificial sequence
  Low Risk Light Chain vs. VK6 Subgroup 2-1-(1) A14
DIVLTQSPAFLSVTPGEKVTFTCQASQSIGTSIHWYQQKTDQSPRLLIKYASESISGIPSRFSGSGSGTDFTLTISSVEAEDAADYYCQQINSWPTTFGGGTKLEIKRT

SEQ ID NO: 52 - DNA, 327 bp, Artificial sequence
  Low Risk + Moderate Risk Light Chain vs. VK6 Subgroup 2-1-(1) A14 DNA SEQ
gacatagttctcacacaatcaccagcattcctctcagttacacccggcgaaaaagtaacctttacctgtcaggcttctcaatctatcggcacttctattcactggtatcaacaaaaaaccgatcaagctcctaaactcctcataaaatacgcatccgaatccatctccggtatcccctccagattttcaggctccggctccggcacagatttcacccttaccattagctcagttgaagccgaagacgcagctgattactactgtcaacaaataaactcatggcccactactttcggcggcggcactaaactcgaaataaaacgtacg

SEQ ID NO: 53 - protein, 109 aa, Artificial sequence
  Low Risk + Moderate Risk Light Chain vs. VK6 Subgroup 2-1-(1) A14
DIVLTQSPAFLSVTPGEKVTFTCQASQSIGTSIHWYQQKTDQAPKLLIKYASESISGIPSRFSGSGSGTDFTLTISSVEAEDAADYYCQQINSWPTTFGGGTKLEIKRT

SEQ ID NO: 54 - protein, 100 aa, Homo sapiens
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHR

SEQ ID NO: 55 - protein, 98 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 56 - protein, 98 aa, Homo sapiens
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCAR

SEQ ID NO: 57 - protein, 98 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 58 - protein, 101 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAR

SEQ ID NO: 59 - protein, 98 aa, Homo sapiens
QVQLVQSGSELKKPGASVKVSCKASGYTFTSYAMNWVRQAPGQGLEWMGWINTNTGNPTYAQGFTGRFVFSLDTSVSTAYLQICSLKAEDTAVYYCAR

SEQ ID NO: 60 - protein, 58 aa, Mus musculus
DVQLQESGPGLVKPSQSLSLTCTVTDYSITSDYAWNWIRQFPGNKLEWMGYISYSGST

SEQ ID NO: 61 - protein, 59 aa, Homo sapiens
  misc_feature (1)..(1): Xaa can be any naturally occurring amino acid
XVQLVQSGAEVKKPGXSVKVSCKASGYTFXSYXIXWVRQAPGQGLEWMGXIXPYXXGXT

SEQ ID NO: 62 - protein, 62 aa, Homo sapiens
  misc_feature (23)..(23): Xaa can be any naturally occurring amino acid
QVQLQESGPGLVKPSQTLSLTCXVSGXSXSSXXXXXXWIRQPPGKGLEWIGXIYYRAXXGXT

SEQ ID NO: 63 - protein, 60 aa, Homo sapiens
  misc_feature (31)..(31): Xaa can be any naturally occurring amino acid
EVQLVESGGGLVQPGGSLRLSCAASGFTFSXYXMXWVRQAPGKGLEWVXXIXXKXXGXXT

SEQ ID NO: 64 - protein, 58 aa, Homo sapiens
QVQLVQSGAEVKKPGASVKVSCKASGYTFTGYYMHWVRQAPGQGLEWMGWINPNSGGT

SEQ ID NO: 65 - protein, 59 aa, Homo sapiens
QITLKESGPTLVKPTQTLTLTCTFSGFSLSTSGVGVGWIRQPPGKALEWLALIYWNDDK

SEQ ID NO: 66 - protein, 58 aa, Homo sapiens
EVQLVESGGGLVQPGGSLRLSCAASGFTFSSYWMSWVRQAPGKGLEWVANIKQDGSEK

SEQ ID NO: 67 - protein, 58 aa, Homo sapiens
QVQLQESGPGLVKPSGTLSLTCAVSGGSISSSNWWSWVRQPPGKGLEWIGEIYHSGST

SEQ ID NO: 68 - protein, 58 aa, Homo sapiens
EVQLVQSGAEVKKPGESLKISCKGSGYSFTSYWIGWVRQMPGKGLEWMGIIYPGDSDT

SEQ ID NO: 69 - protein, 61 aa, Homo sapiens
QVQLQQSGPGLVKPSQTLSLTCAISGDSVSSNSAAWNWIRQSPSRGLEWLGRTYYRSKWYN

SEQ ID NO: 70 - protein, 58 aa, Homo sapiens
QVQLVQSGSELKKPGASVKVSCKASGYTFTSYAMNWVRQAPGQGLEWMGWINTNTGNP

SEQ ID NO: 71 - protein, 60 aa, Mus musculus
SYNPSLKSRISITRDTSKNQFFLQLNSVTTEDTATYYCASFDYAHAMDYWGQGTSVTVSS

SEQ ID NO: 72 - protein, 70 aa, Homo sapiens
  misc_feature (14)..(14): Xaa can be any naturally occurring amino acid
NYAQKFQGRVTITXDXSTSTAYMELSSLRSXDTAVYYCARXXXXXXXXXXXXXDXXFDXWGQGTLVTVSS

SEQ ID NO: 73 - protein, 70 aa, Homo sapiens
  misc_feature (1)..(1): Xaa can be any naturally occurring amino acid
XYNPSLKSRVTISVDTSKNQFSLXLXSVTAADTAVYYCARXXXXXXXXXXXXXXXXFDXWGQGTXVTVSS

SEQ ID NO: 74 - protein, 70 aa, Homo sapiens
  misc_feature (40)..(52): Xaa can be any naturally occurring amino acid
YYADSVKGRFTISRDNSKNTLYLQMNSLRAEDTAVYYCAXXXXXXXXXXXXXYYXXFDXWGQGTLVTVSS

SEQ ID NO: 75 - protein, 70 aa, Homo sapiens
  misc_feature (41)..(55): Xaa can be any naturally occurring amino acid
NYAQKFQGRVTMTRDTSISTAYMELSRLRSDDTAVYYCARXXXXXXXXXXXXXXXYFDYWGQGTLVTVSS

SEQ ID NO: 76 - protein, 70 aa, Homo sapiens
  misc_feature (42)..(55): Xaa can be any naturally occurring amino acid
RYSPSLKSRLTITKDTSKNQVVLTMTNMDPVDTATYYCAHRXXXXXXXXXXXXXXYFDYWGQGTLVTVSS

SEQ ID NO: 77 - protein, 70 aa, Homo sapiens
  misc_feature (41)..(55): Xaa can be any naturally occurring amino acid
YYVDSVKGRFTISRDNAKNSLYLQMNSLRAEDTAVYYCARXXXXXXXXXXXXXXXYFDYWGQGTLVTVSS

SEQ ID NO: 78 - protein, 70 aa, Homo sapiens
  misc_feature (41)..(55): Xaa can be any naturally occurring amino acid
NYNPSLKSRVTISVDKSKNQFSLKLSSVTAADTAVYYCARXXXXXXXXXXXXXXXYFDYWGQGTLVTVSS

SEQ ID NO: 79 - protein, 70 aa, Homo sapiens
  misc_feature (41)..(55): Xaa can be any naturally occurring amino acid
RYSPSFQGQVTISADKSISTAYLQWSSLKASDTAMYYCARXXXXXXXXXXXXXXXYFDYWGQGTLVTVSS

SEQ ID NO: 80 - protein, 70 aa, Homo sapiens
  misc_feature (41)..(55): Xaa can be any naturally occurring amino acid
DYAVSVKSRITINPDTSKNQFSLQLNSVTPEDTAVYYCARXXXXXXXXXXXXXXXYFDYWGQGTLVTVSS

SEQ ID NO: 81 - protein, 70 aa, Homo sapiens
  misc_feature (41)..(55): Xaa can be any naturally occurring amino acid
TYAQGFTGRFVFSLDTSVSTAYLQICSLKAEDTAVYYCARXXXXXXXXXXXXXXXYFDYWGQGTLVTVSS

SEQ ID NO: 82 - DNA, 1404 bp, Homo sapiens
atgggatggagttgcattatacttttcctcgttgccaccgccactggagttcactctgacgtacaacttcaagaatctggcccaggtctcgtcaaaccttctcaaactctctcactcacctgcactgttactgactactctattacatccgactacgcttggaactggatccgacaatttcctggtaaaaaactcgaatggatgggttatatttcttactctggctccacctcctacaatccttctctgaaatcacgcatcacaatttcccgcgatacctctaaaaatcaattttcactccaactcaattctgttaccgccgccgatactgccacctactactgtgcctcttttgactacgctcacgccatggattattggggacagggtactaccgttaccgtaagctcagccagcacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 83 - protein, 467 aa, Homo sapiens
MGWSCIILFLVATATGVHSDVQLQESGPGLVKPSQTLSLTCTVTDYSITSDYAWNWIRQFPGKKLEWMGYISYSGSTSYNPSLKSRITISRDTSKNQFSLQLNSVTAADTATYYCASFDYAHAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - DNA, 3985 bp, Homo sapiens
  CDS (293)..(3211)
  sig_peptide (293)..(349)
  mat_peptide (350)..(3208)
  misc_feature (374)..(598): Immunoglobulin
gaagggcagacagagtgtccaaaagcgtgagagcacgaagtgaggagaaggtggagaagagagaagaggaagaggaagaggaagagaggaagcggagggaactgcggccaggctaaaaggggaagaagaggatcagcccaaggaggaggaagaggaaaacaagacaaacagccagtgcagaggagaggaacgtgtgtccagtgtcccgatccctgcggagctagtagctgagagctctgtgccctgggcaccttgcagccctgcacctgcctgccacttccccaccgaggccatgggcccaggagttctgctgctcctgctggtggccacagcttggcatggtcagggaatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctgaggagttgacgacagggagtaccactctcccctcctccaaacttcaactcctccatggatggggcgacacggggagaacatacaaactctgccttcggtcatttcactcaacagctcggcccagctctgaaacttgggaaggtgagggattcaggggaggtcagaggatcccacttcctgagcatgggccatcactgccagtcaggggctgggggctgagccctcacccccccctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctcacaggacctcttagtctctgccctatacgccgccttcactccacagcctcacccctcccacccccatactggtactgctgtaatgagccaagtggcagctaaaagttgggggtgttctgcccagtcccgtcattctgggctagaaggcaggggaccttggcatgtggctggccacaccaagcaggaagcacaaactcccccaagctgactcatcctaactaacagtcacgccgtgggatgtctctgtccacattaaactaacagcattaatgca

SEQ ID NO: 85 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 86 - DNA, 327 bp, Homo sapiens
gaaatagtccttacccaatctcccggaaccctctcagtatctcccggcgaacgagtaaccttttcatgtagagcatcccaatccatcggcacttcaattcactggtatcagcagaaaacaggtcaatccccacggcttcttataaaatatgcatcagaatcaatttctggcatcccagacagattttcaggttcaggatcaggcaccgatttcacacttacaatatccagagtcgaatcagaagattttgcagattactattgtcaacaaataaacagctggcccactacattcggacaaggcacaaaactcgaaattaaacgtacg

SEQ ID NO: 87 - protein, 109 aa, Homo sapiens
EIVLTQSPGTLSVSPGERVTFSCRASQSIGTSIHWYQQKTGQSPRLLIKYASESISGIPDRFSGSGSGTDFTLTISRVESEDFADYYCQQINSWPTTFGQGTKLEIKRT

SEQ ID NO: 88 - DNA, 327 bp, Homo sapiens
gaaatagttcttactcaatcccccggtacactctcagtttccccaggcgaacgcgtcactttttcttgcagagcatcacaatcaatcggcacttcaattcattggtatcaacaaaaaacaggacaggccccacgacttcttattaaatatgcatcagaatcaatttctggcatcccagacagattttcaggttcaggatcaggcaccgatttcacacttacaatatccagagtcgaatcagaagattttgcagattactattgtcaacaaataaacagctggcccactacattcggacaaggcacaaaactcgaaattaaacgtacg

(19) United States
(12) Patent Application Publication
(10) Pub. No.: US 2009/0246208 A1
(43) Pub. Date: Oct. 1, 2009

(54) METHODS FOR PREVENTING AND TREATING CANCER METASTASIS AND BONE LOSS ASSOCIATED WITH CANCER METASTASIS

(75) Inventors: Michael Kavanaugh, Orinda, CA (US); Cheng Liu, Richmond, CA (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignees: Novartis AG, Basel (CH); Xoma Technology Ltd., Hamilton (BM)

(21) Appl. No.: 12/159,665

(22) PCT Filed: Jan. 4, 2007
(86) PCT No.: PCT/US07/00405

§ 371 Date: Sep. 8, 2008

Related U.S. Application Data
(60) Provisional application No. 60/756,944, filed on Jan. 5, 2006.

(51) Int. Cl. A61K 39/395; A61P 19/00

(52) U.S. Cl. 424/158.1

(57) ABSTRACT

Methods for preventing and treating osteolysis, cancer metastasis and bone loss associated with cancer metastasis by administering an M-CSF-antagonist in combination with a therapeutic agent to a subject are provided.

### TECHNICAL FIELD

This invention relates to methods for preventing and treating osteolysitic diseases, including cancer metastasis and bone loss associated with cancer metastasis by administering an M-CSF-antagonist in combination with another therapeutic agent to a subject.

### BACKGROUND OF THE INVENTION

Osteoclasts, which mediate bone resorption, are involved in normal and abnormal bone remodeling processes, including osteolytic diseases. Osteoclasts are multinucleated cells differentiating from haemopoietic cells. It is generally accepted that osteoclasts are formed by the fusion of mononuclear precursors derived from haemopoietic stem cells in the bone marrow, rather than incomplete cell divisions (Chambers, Bone and Mineral Research, 6: 1-25, 1989; Göthling et al., Clin Orthop Relat R. 120: 201-228, 1976; Kahn et al., Nature 258: 325-327, 1975, Suda et al., Endocr Rev 13: 66-80, 1992; Walker, Science 180: 875, 1973; Walker, Science 190: 785-787, 1975; Walker, Science 190: 784-785, 1975). They share a common stem cell with monocyte-macrophage lineage cells (Ash et al., Nature 283: 669-670, 1980, Kerby et al., J. Bone Miner Res 7: 353-62, 1992). The differentiation of osteoclast precursors into mature multinucleated osteoclasts requires different factors including hormonal and local stimuli (Athanasou et al., Bone Miner 3: 317-333, 1988; Feldman et al., Endocrinology 107: 1137-1143, 3980; Walker, Science 190: 784-785, 1975; Zheng et al., Histochem J 23:180-188, 1991) and living bone and bone cells have been shown to play a critical role in osteoclast development (Hagenaars et al., Bone Miner 6: 179-189, 1989). Osteoblastic or bone marrow stromal cells are also required for osteoclast differentiation. One of the factors produced by these cells that supports osteoclast formation is macrophage-colony stimulating factor, M-CSF (Wiktor-Jedrzejczak et al., Proc Natl Acad Sci USA 87: 4828-4832, 1990; Yoshida et al., Nature 345: 442-444, 1990). Receptor activator for NF-κB ligand (RANKL, also known as TRANCE, ODF and OPGL) is another signal (Suda et al., Endocr Rev 13: 66-80, 1992) through which osteoblastic/stromal cells stimulate osteoclast formation and resorption via a receptor, RANK (TRANCER), located on osteoclasts and osteoclast precursors (Lacey et al., Cell 93: 165-176, 1998; Tsuda et al., Biochem Biophys Res Co 234: 137-142, 1997; Wong et al., J Exp Med 186: 2075-2080, 1997; Wong et al., J Biol. Chem. 272: 25190-25194, 1997; Yasuda et al., Endocrinology 139: 1329-1337, 1998; Yasuda et al., Proc Natl Acad Sci US 95: 3597-3602a 1998). Osteoblasts also secrete a protein that strongly inhibits osteoclast formation called osteoprotegerin (OPG, also known as OCIF), which acts as a decoy receptor for the RANKL thus inhibiting the positive signal between osteoclasts and osteoblasts via RANK and RANKL.

Osteoclasts are responsible for dissolving both the mineral and organic bone matrix (Blair et al., J Cell Biol 102: 1164-1172, 1986). Osteoclasts represent terminally differentiated cells expressing a unique polarized morphology with specialized membrane areas and several membrane and cytoplasmic markers, such as tartrate resistant acid phosphatase (TRAP) (Anderson et al. 1979), carbonic anhydrase II (Väänänen et al., Histochemistry 78: 481-485, 1983), calcitonin receptor (Warshafsky et al., Bone 6: 179-185, 1985) and vitronectin receptor (Davies et al., J Cell Biol 109: 1817-1826, 1989). Multinucleated osteoclasts usually contain less than 10 nuclei, but they may contain up to 100 nuclei being between 10 and 100 μm in diameter (Göthling et al., Clin Orthop Relat R 120: 201-228, 1976). This makes them relatively easy to identify by light microscopy. They are highly vacuolated when in the active state, and also contain many mitochondria, indicative of a high metabolic rate (Mundy, in Primer on the metabolic bone diseases and disorders of mineral metabolism, pages 18-22, 1990). Since osteoclasts play a major role in osteolytic bone metastases, there is a need in the art for new agents and methods for preventing osteoclast stimulation and function.

Cancer metastasis is the primary cause of post-operation or post-therapy recurrence in cancer patients. Despite intensive efforts to develop treatments, cancer metastasis remains substantially refractory to therapy. Bone is one of the most common sites of metastasis of various types of human cancers (e.g., breast, lung, prostate and thyroid cancers). The occurrence of osteolytic bone metastases causes serious morbidity due to intractable pain, high susceptibility to fracture, nerve compression and hypercalcemia. Despite the importance of these clinical problems, there are few available treatments for bone loss associated with cancer metastasis.

Several therapeutic strategies targeting osteolytic disease are currently being used or under development, where efforts have mainly focused on the development of drugs to block bone resorption through inhibiting the formation or activity of osteoclasts. The bisphosphonates (BPs), pyrophosphate analogs that concentrate in bone, are to date the most effective inhibitor of bone resorption. BPs are taken up by osteoclasts, inhibiting their activity and causing the cells to undergo apoptosis, thereby inhibiting bone resorption. Alendronate was the first BP inhibitor of bone resorption to show a significant reduction in spine/hip fractures, and is approved for treatment of osteoporosis. The latest generation BP, Zometa, is approved for treatment of hypercalcemia and bone disease in solid tumors and multiple myeloma and is under investigation for possible treatment of Paget's disease and bone metastasis resulting from solid tumors and multiple myeloma. Zometa acts at very low doses, and is given as a15 minute i.v. infusion once a month, but also affects osteoblasts and may cause side effects such as renal toxicity and osteonecrosis of the jaw (Fromigue and Brody, J, Endocrinol. Invest. 25:39-46, 2002; Ibrahim, A. et al., Clin. Canc. Res. 9:2394-99, 2003; Body, J. J., The Breast. S2:S37-44, 2003; Yaccoby, S. et al., Brit. J. Hemat., 116:278-80, 2002; Corey, E. et al., Clin. Canc. Res. 9: 295-306, 2003; Coleman, R. E., Sem. Oncol., 29(6): 43-49, 2002; Coleman, R. E., Eur. Soc. Med. Oncol. 16:687-95, 2005; Bamias et al., J Clin Oncol 13: 8580-8587, 2005. Thus, there remains a need in the art to identify new agents and methods for preventing or treating osteolytic diseases and/or cancer metastasis, including osteolytic bone metastases.

### SUMMARY OF THE INVENTION

The compositions and methods of the present invention fulfill the aforementioned and other related needs in the art. In one embodiment of the invention, a method is provided for treating a subject suffering from or at risk of an osteolytic disorder comprising administering to the subject a monotherpaeutically amount of a M-CSF antagonist and a monotherapeutically effective amount of a second anti-osteoclast agent for a transition period of about 1 day to a year, during which the M-CSF antagonist reduces the number of active osteoclasts to a therapeutically desirable level. Exemplary M-CSF antagonists include M-CSF antibodies and exemplary second anti-osteoclast agents include bisphosphonates and RANKL inhibitors, including anti-RANKL antibodies. The methods and/or uses involving anti-M-CSF antibody and osteoclast inhibitor herein optionally exclude the use of RX1, 5H4, MC1 and MC3-derived antibodies disclosed in International Publication No. WO 2005/068503. The duration of the transition period may be, for example, at least one-day up to one year, and may be monitored, e.g., by relevant markers of osteoclast growth or activity. Alternatively, they may be given simultaneously.

By way of example, markers of bone formation include but are not limited to calcium, and total and bone-specific alkaline phosphatase (BAP), osteocalcin (OC, bone gla-protein), Procollagen type I C propeptide (PICP), Procollagen type 1 N propeptide (PINP), and markers of bone resorption include but are not limited to NTX (N-terminal cross-linking telopeptide of bone collagen) and CTX (C-terminal cross-linking telopeptide of bone collagen), pyridinium crosslinks (pyridinoline and deoxypyridinoline [DPD]) and associated peptides, bone type I collagen degradation products hydroxyproline and hydroxylysine glycosides, tartrate-resistant acid phosphatase (TRACP), and bone sialoprotein (BSP). See Fohr et al., J. Clin. Endocrinol. Metab., November 2003, 88(11):5059-5075.

In related embodiments, the aforementioned methods are provided wherein the second anti-osteoclast agent is discontinued after the transition period. In other related embodiments, the aforementioned methods are provided wherein the amount of the second anti-osteoclast agent is reduced after the transition period. In further related embodiments, the aforementioned methods are provided wherein the amount of M-CSF antagonist is reduced after the transition period.

It is contemplated that the methods of the instant invention achieve their therapeutic potential by inhibiting the interaction between M-CSF and its receptor (M-CSFR). It is further contemplated that the inhibition of M-CSF/M-CSFR interaction inhibits osteoclast proliferation and/or differentiation. In any of the methods or compositions of the invention, the M-CSF antagonist may be a polypeptide comprising an anti-M-CSF antibody; a polypeptide comprising an anti-M-CSFR antibody; a soluble polypeptide comprising an M-CSF mutein or derivative thereof; or a soluble polypeptide comprising an M-CSFR mutein or derivative thereof; or a nucleic acid molecule that inhibits the expression of M-CSF or M-CSFR. The identification, production and modification of various M-CSF antagonists is described in Int'l Publication No. WO 2005/068503, hereby incorporated by reference in its entirety.

The M-CSF antibody may be a polyclonal antibody; a monoclonal antibody; a humanized antibody; a human antibody; a human engineered antibody; a chimeric antibody; Fab, F(ab′)₂ or Fᵥ antibody fragment; or a mutein of any one of the aforementioned antibodies.

M-CSF antibodies of the instant invention that inhibit osteolysis are described in Int'l Publication No. WO 2005/068503, which is hereby incorporated by reference in its entirety for its teaching with respect to M-CSF antibodies.

In one embodiment of the invention, a non-murine monoclonal antibody is provided, including functional fragment, that specifically binds to the same epitope of M-CSF as anyone of murine monoclonal antibody RX1, MC1, or MC3 having the amino acid sequences set forth in FIGS. 1, 3, and 4, respectively. In a related embodiment, an aforementioned antibody is provided wherein the antibody is selected from the group consisting of a polyclonal antibody; a monoclonal antibody including a Human Engineered™ antibody; a humanized antibody; a human antibody; a chimeric antibody; Fab, F(ab′)2; Fv; Sc Fv or SCA antibody fragment; a diabody; linear antibody; or a mutein of any one of these antibodies, that preferably retain binding affinity of at least 10⁻⁷, 10⁻⁸ or 10⁻⁹ or higher. A non-murine monoclonal antibody, including functional fragment, that competes with monoclonal antibody RX1, MC1, and/or MC3 having the amino acid sequence set forth in FIG. 1 for binding to M-CSF by more than 75%, is also contemplated.

In another embodiment, a non-murine monoclonal antibody, including functional fragment, wherein said non-murine monoclonal antibody or functional fragment thereof binds an epitope of M-CSF that includes at least 4, 5, 6, 7 or 8 contiguous residues of amino acids 98-105 of FIG. 7 is provided.

In another embodiment, the invention provides a non-murine monoclonal antibody, including functional fragment, wherein said non-murine monoclonal antibody or functional fragment thereof binds an epitope of M-CSF that includes at least 4, 5, 6, 7 or 8 contiguous residues of amino acids 65-73 or 138-144 of FIG. 7 corresponding to M-CSF epitopes recognized by 5H4 or MC3).

In yet another embodiment, the aforementioned antibody or fragment that binds an epitope of M-CSF that includes amino acids 98-105 of FIG. 7 is provided. In a related embodiment, the aforementioned antibody is provided comprising CDR3 of FIG. 1A. In another embodiment, the antibody is provided comprising at least 1, 2, 3, 4, 5, or 6 CDRs of murine antibody RX1 set forth in FIG. 1A. Such an antibody that comprises at least 1, 2, 3, 4, or 5 CDRs of murine antibody RX1 may also comprise at least 1, 2, 3, 4, or 5 CDRs of any of the 6 CDRs of antibody 5H4 set forth in FIG. 8A-B. Alternatively, the antibody that comprises at least 1, 2, 3, 4, or 5 CDRs of murine antibody RX1 may also comprise at least 1, 2, 3, 4, or 5 CDRs of any of the 6 CDRs of antibody MC1 set forth in FIG. 8A-B. In yet another alternative, the aforementioned antibody may also comprise at least 1, 2, 3, 4, or 5 CDRs of any of the 6 CDRs of antibody MC3 set forth in FIG. 8A-B. In a related embodiment, the antibody that comprises at least 1, 2, 3, 4, or 5 CDRs of murine antibody RX1 may comprise at least 1, 2, 3, 4 or 5 CDRs of the consensus CDRs-set forth in FIG. 8A-D is provided. In still another related embodiment, in the aforementioned antibody one or more residues of the consensus CDR(s) is substituted by the corresponding residue of any of the CDRs of antibody murine RX1, 5H4, MC1 or MC3. The desired binding affinity may be retained even though one or more of the amino acids in the antibody have been mutated, e.g. by conservative substitutions in the CDRs, and/or conservative or non-conservative changes in the low and moderate risk residues.

In another embodiment of the invention, variants of the aforementioned antibody are provided, comprising a variable heavy chain amino acid sequence which is at least 60, 65, 70, 75, 80, 85, 90, 91, 92, 93, 94, 95, 96, 97, 98, or 99% homologous to the amino acid sequence set forth in FIG. 1A, 2, 3, or 4. In a related embodiment, the antibody comprises a variable light chain amino acid sequence which is at least 60, 65, 70, 75, 80, 85, 90, 91, 92, 93, 94, 95, 96, 97, 98, or 99% homologous to the amino acid sequence set forth in FIG. 1A, 2, 3, or 4.

In yet another embodiment, the antibody comprises a constant region and one or more heavy and light chain variable framework regions of a human antibody sequence. In a related embodiment, the antibody comprises a modified or unmodified constant region of a human IgG1, IgG2, IgG3 or IgG4. In a preferred embodiment, the constant region is human IgG1 or IgG4, which may optionally be modified to enhance or decrease certain properties. In the case of IgG1, modifications to the constant region, particularly the hinge or CH2 region, may increase or decrease effector function, including ADCC and/or CDC activity. In other embodiments, an IgG2 constant region is modified to decrease antibody-antigen aggregate formation. In the case of IgG4, modifications to the constant region, particularly the hinge region, may reduce the formation of half-antibodies.

In one embodiment of the invention, a non-murine monoclonal antibody is provided that specifically binds to the same epitope of M-CSF as any one of the murine antibodies RX1, 5H4, MC1 or MC3 as described in Int'l Publication No. WO 2005/068503, or competes with any one of the aforementioned murine antibodies for binding to M-CSF by more than 10%, more preferably by more than 25%, still more preferably by more than 50%, even more preferably by more than 75%, and most preferably more than 90%. Antibodies derived from the sequences of such murine antibodies, including chimeric, human, humanized, human engineered antibodies, or fragments or muteins or chemically derivatized versions thereof, are described in WO 2005/068503.

The term “RX1-derived antibody” includes any one of the following:

1) an amino acid variant of murine antibody RX1 having the amino acid sequence set out in FIG. 1, including variants comprising a variable heavy chain amino acid sequence which is at least 60, 65, 70, 75; 80, 85, 90, 91, 92, 93, 94, 95, 96, 97, 98, or 99% homologous to the amino acid sequence as set forth in FIG. 1, and/or comprising a variable light chain amino acid sequence which is at least 60, 65, 70, 75, 80, 85, 90, 91, 92, 93, 94, 95, 96, 97, 98, or 99% homologous to the amino acid sequence as set forth in FIG. 1, taking into account similar amino acids for the homology determination;

2) M-CSF-binding polypeptides (excluding murine antibody RX 1) comprising one or more complementary determining regions (CDRs) of murine antibody RX 1 having the amino acid sequence set out in FIG. 1, preferably comprising at least CDR3 of the RX1 heavy chain, and preferably comprising two or more, or three or more, or four or more, or five or more, or all six CDRs;

3) Human Engineered™ antibodies having the heavy and light chain amino acid sequences set out in FIGS. 9B through 12B or variants thereof comprising a heavy or light chain having at least 60% amino acid sequence identity with the original Human Engineered™ heavy or the light chain of FIGS. 9B through 12B, more preferably at least 80%, more preferably at least 85%, more preferably at least 90%, and most preferably at least 95%, including for example, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% identical;

4) M-CSF-binding polypeptides (excluding murine antibody RX1) comprising the high risk residues of one or more CDRs of the Human Engineered™ antibodies of FIGS. 9B through 12B, and preferably comprising high risk residues of two or more, or three or more, or four or more, or five or more, or all six CDRs;

5) Human Engineered™ antibodies or variants retaining the high risk amino acid residues set out in FIG. 1B, and comprising one or more changes at the low or moderate risk residues set out in FIG. 1B;
for example, comprising one or more changes at a low risk residue and conservative substitutions at a moderate risk residue set out in FIG. 1B, or
for example, retaining the moderate and high risk amino acid residues set out in FIG. 1B and comprising one or more changes at a low risk residue,
where changes include insertions, deletions or substitutions and may be conservative substitutions or may cause the engineered antibody to be closer in sequence to a human light chain or heavy chain sequence, a human germline light chain or heavy chain sequence, a consensus human light chain or heavy chain sequence, or a consensus human germline light chain or heavy chain sequence;

that retain ability to bind M-CSF. Such antibodies preferably bind to M-CSF with an affinity of at least 10⁻⁷, 10⁻⁸ or 10⁻⁹ or higher and preferably neutralize the osteoclastogenesis inducing activity of M-CSF.

Similarly, the term “MC3-derived antibody” includes any one of the following:

1) an amino acid variant of murine antibody MC3 having the amino acid sequence set out in FIG. 4, including variants comprising a variable heavy chain amino acid sequence which is at least 60, 65, 70, 75, 80, 85, 90, 91, 92, 93, 94, 95, 96, 97, 98, or 99% homologous to the amino acid sequence as set forth in FIG. 4, and/or comprising a variable light chain amino acid sequence which is at least 60, 65, 70, 75, 80, 85, 90, 91, 92, 93, 94, 95, 96, 97, 98, or 99% homologous to the amino acid sequence as set forth in FIG. 4, taking into account similar amino acids for the homology determination;

2) M-CSF-binding polypeptides (optionally including or excluding murine antibody MC3) comprising one or more complementary determining regions (CDRs) of murine antibody MC3 having the amino acid sequence set out in FIG. 4, preferably comprising at least CDR3 of the MC3 heavy chain, and preferably comprising two or more, or three or more, or four or more, or five or more, or all six CDRs;

3) Human Engineered™ antibodies generated by altering the murine sequence according to the methods set forth in Studnicka et al., U.S. Pat. No. 5,766,886 and Example 4A herein, using the Kabat numbering set forth in FIGS. 13C-13E to identify low, moderate and high risk residues; such antibodies comprising at least one of the following heavy chains and at least one of the following light chains: (a) a heavy chain in which all of the low risk residues have been modified, if necessary, to be the same residues as a human reference immunoglobulin sequence or (b) a heavy chain in which all of the low and moderate risk residues have been modified, if necessary, to be the same residues as a human reference immunoglobulin sequence, (c) a light chain in which all of the low risk residues have been modified, if necessary, to be the same residues as a human reference immunoglobulin sequence or (b) a light chain in which all of the flow and moderate risk residues have been modified, if necessary, to be the same residues as a human reference immunoglobulin sequence

4) variants of the aforementioned antibodies in preceding paragraph (3) comprising a heavy or light chain having at least 60% amino acid sequence identity with the original Human Engineered™ heavy or the light chain, more preferably at least 80%, more preferably at least 85%, more preferably at least 90%, and most preferably at least 95%, including for example, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% identical;

5) M-CSF-binding polypeptides (optionally including or excluding murine antibody MC3) comprising the high risk residues of one or more CDRs of the murine MC3 antibody of FIG. 4, and preferably comprising high risk residues of two or more, or three or more, or four or more, or five or more, or all six CDRs;

6) Human Engineered™ antibodies or variants retaining the high risk amino acid residues of murine MC3 antibody, and comprising one or more changes at the low or moderate risk residues;
for example, comprising one or more changes at a low risk residue and conservative substitutions at a moderate risk residue, or
for example, retaining the moderate and high risk amino acid residues and comprising one or more changes at a low risk residue,
where changes include insertions, deletions or substitutions and may be conservative substitutions or may cause the engineered antibody to be closer in sequence to a human light chain or heavy chain sequence, a human germline light chain or heavy chain sequence, a consensus human light chain or heavy chain sequence, or a consensus human germline light chain or heavy chain sequence;

that retain ability to bind M-CSF. Such antibodies preferably bind to M-CSF with an affinity of at least 10⁻⁷, 10⁻⁸ or 10⁻⁹ or higher and preferably neutralize the osteoclastogenesis inducing activity of M-CSF.

The term “5H4-derived antibody” or “MC1-derived antibody” is similarly defined according to the above description.

As described in detail herein, RX1, 5H4, MC1 or MC3-derived antibodies, including Human Engineered™ antibodies or variants, may be of different isotypes, such as IgG, IgA, IgM or IgE. Antibodies of the IgG class may include a different constant region, e.g. an IgG2 antibody may be modified to display an IgG1 or IgG4 constant region. In preferred embodiments, the invention provides Human Engineered antibodies or variants comprising a modified or unmodified IgG1 or IgG4 constant region. In the case of IgG1, modifications to the constant region, particularly the binge or CH2 region, may increase or decrease effector function, including ADCC and/or CDC activity. In other embodiments, an IgG2 constant region is modified to decrease antibody-antigen aggregate formation. In the case of IgG4, modifications to the constant region, particularly the hinge region, may seduce the formation of half-antibodies. In specific exemplary embodiments, mutating the IgG4 hinge sequence Cys-Pro-Ser-Cys to the IgG1 hinge sequence Cys-Pro-Pro-Cys is provided.

A pharmaceutical composition comprising any one of the aforementioned M-CSF antagonists or M-CSF antibodies and a pharmaceutically acceptable carrier, excipient or diluent may be administered according to the present invention.

It may be further advantageous to mix two or more M-CSF antagonists together or to co-administer an M-CSF antagonist and a second anti-osteoclast agent to provide improved efficacy against osteolytic disorders of the invention, including cancer metastasis and/or bone loss associated with cancer metastasis.

In exemplary embodiments of the invention, the aforementioned methods are provided wherein the second anti-osteoclast agent is a bisphosphonate. In a further embodiment, the bisphosphonate is zoledronate, pamidronate, clodronate, etidronate, tiludronate, alendronate, ibandronate or risedronate. Exemplary other anti-osteoclast agents include bisphosphonates, PTHrP neutralizing agents (e.g., antibody, antisense, siRNA), cathepsin K inhibitors, MIP-1-α antagonists, RANK/RANKL neutralizing agents (e.g., anti-RANK antibody, anti-RANKL antibody, or antisense, soluble RANKL receptor or muteins thereof), RANKL vaccine, osteoprotegrin (OPG), platelet-derived growth factors (PDGF), src kinase inhibitors, gallium maltolate, and matrix metalloproteinase (MMP) inhibitors.

The therapeutic methods of the present invention may be combined with yet a third therapeutic agent such as a cancer chemotherapeutic agent or with radiation treatment or surgery. Cancer chemotherapeutic agents include, without limitation, alkylating agents, such as carboplatin and cisplatin; nitrogen mustard alkylating agents; nitrosourea alkylating agents, such as carmustine (BCNU); antimetabolites, such as methotrexate; purine analog antimetabolites, mercaptopurine; pyrimidine analog antimetabolites, such as fluorouracil (5-FU) and gemcitabine; hormonal antineoplastics, such as goserelin, leuprolide, and tamoxifen; natural antineoplastics, such as aldesleukin, interleukin-2, docetaxel, etoposide (VP-16), interferon alfa, paclitaxel, and tretinoin (ATRA); antibiotic natural antineoplastics, such as bleomycin, dactinomycin, daunorubicin, doxorubicin, and mitomycin; and vinca alkaloid natural antineoplastics, such as vinblastine, vincristine, vindesine; hydroxyurea; aceglatone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide, Schizophyllan, cytarabine, dacarbazine, thioinosine, thiotepa, tegafur, neocarzinostatin, OK-432, bleomycin, furtulon, broxuridine, busulfan, honvan, peplomycin, Bestatin (ubenimex), interferon-β, mepitiostane, mitobronitol, merphalan, laminin peptides, lentinan, Coriolus versicolor extract, tegafur/uracil, estramustine (estrogen/mechlorethamine).

Further, additional agents used as adjunctive therapy for cancer patients include EPO, G-CSF, ganciclovir; antibiotics, leuprolide; meperidine; zidovudine (AZT); interleukins 1 through 18, including mutants and analogues; interferons or cytokines, such as interferons α, β, and γ; hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; anti-angiogenesis factors; antigenic materials; pro-drugs; growth factor receptor kinase inhibitors; anti-Her2 antibody; and VEGF neutralizing antibody.

Subsequent to the transition, period, the amount of M-CSF antagonist or amount of second anti-osteoclast agent required to achieve a therapeutic effect may be reduced. Thus, after such time period, an M-CSF antagonist may improve efficacy of the second anti-osteoclast agent, or reduce side effects associated with administration of the second anti-osteoclast agent, or improve the safety of the second anti-osteoclast agent. An M-CSF antagonist may also improve efficacy, reduce side effects of, or improve safety of a third therapeutic modality such as cancer chemotherapy, other adjunctive therapy, surgery or radiation therapy. In another embodiment of the invention, a package, vial or container is provided comprising a medicament comprising an M-CSF antagonist and instructions that the medicament should be used in combination with a second and/or third therapeutic agent and/or with surgery or radiation therapy.

Numerous osteolytic disorders are contemplated to be amenable to treatment according to the present invention. As used herein, an “osteolytic disorder” is any condition resulting from increased osteoclast activity. A subject at risk of an osteolytic disorder may be a subject in a group predisposed to develop an osteolytic disorder, or a subject suffering from a disease that causes or contributes to increased osteoclastic activity. In exemplary embodiments of the invention, the osteolytic disorder may be a metabolic bone disease associated with relatively increased osteoclast activity, including an endocrinopathy (hypercortisolism, hypogonadism, primary or secondary hyperparathyroidism, hyperthyroidism), hypercalcemia, deficiency state (rickets/osteomalacia, scurvy, malnutrition), chronic disease (malabsorption syndromes, chronic renal failure (renal osteodystrophy), chronic liver disease (hepatic osteodystrophy)), drugs (glucocorticoids (glucocorticoid-induced osteoporosis), heparin, alcohol), or hereditary disease (osteogenesis imperfecta, homocystinuria), cancer, osteoporosis, osteopetrosis, inflammation of bone associated with arthritis and rheumatoid arthritis, periodontal disease, fibrous dysplasia, and/or Paget's disease.

In other exemplary embodiments, the osteolytic disorder may be a metastatic cancer to bone, wherein the metastatic cancer is breast, lung, renal, multiple myeloma, thyroid, prostate, adenocarcinoma, blood cell malignancy, including leukemia and lymphoma; head and neck cancer; gastrointestinal cancer, including esophageal cancer, stomach cancer, colon cancer, intestinal cancer, colorectal cancer, rectal cancer, pancreatic cancer, liver cancer, cancer of the bile duct or gall bladder; malignancy of the female genital tract, including ovarian carcinoma, uterine endometrial cancer, vaginal cancer, or cervical cancer; bladder cancer; brain cancer, including neuroblastoma; sarcoma, osteosarcoma; or skin cancer, including malignant melanoma or squamous cell cancer.

In exemplary embodiments of the invention, any of the foregoing methods may prevent or reduce bone loss or preventing or reducing bone metastases or severity of bone loss associated with the disease.

M-CSF antibody administered according to the present invention may be given at a dose between about 2 μg/kg to 30 mg/kg, 0.1 mg/kg to 30 mg/kg or 0.1 mg/kg to 10 mg/kg body weight.

### BRIEF DESCRIPTION OF THE DRAWINGS

FIG. 1A shows the amino acid sequence of M-CSF-specific murine antibody RX1 (SEQ ID NOs: 2 and 4) (encoded by the cDNA insert of the plasmid deposited with the American Type Culture Collection, Manassas, Va., USA, under ATCC deposit number PTA-6113) and a corresponding nucleic acid sequence (SEQ ID NOs: 1 and 3). The CDR regions are numbered and shown in bold.

FIGS. 1B and 1C show the amino acid sequences of M-CSF specific murine antibody RX1) light (SEQ ID NO: 5) and heavy chains (SEQ ID NO: 6), respectively, with high risk (bold), moderate risk (underline), and low risk residues identified according to Studnicka et al., WO93/11794.

FIGS. 2, 3, and 4 show the amino acid sequences of MCSF-specific murine antibodies 5H4 (SEQ ID NOs: 10 and 11), MC1 (SEQ ID NOs: 12 and 13) (produced by the hybridoma deposited under ATCC deposit number PTA-6263) and MC3 (SEQ ID NOs: 14 and 15) (produced by the hybridoma deposited under ATCC deposit number PTA-6264), respectively.

FIG. 5 is the amino acid sequence of M-CSFα (SEQ ID NO: 7).

FIG. 6 is the amino acid sequence of M-CSFβ (SEQ ID NO: 8).

FIG. 7 is the amino acid sequence of M-CSFγ (SEQ ID NO: 9). A number of polymorphisms in the DNA sequence may result in amino acid differences. For example, a common polymorphism provides an Ala rather than Pro at position 104.

FIGS. 8A and B are an alignment of CDR regions of the heavy and light chain amino acid sequences of human M-CSF specific murine antibodies RX1; 5H4; MC1; and MC3 (SEQ ID NOs: 16-38).

FIG. 9A shows (a) the risk line for the murine RX1 heavy chain (H=high risk, M=moderate risk, L=low risk), (b) the RX1 heavy chain amino acid sequence (SEQ ID NO: 6), (c) the amino acid sequence of the closest human consensus sequence, Kabat Vh2 consensus, aligned to RX1 (SEQ ID NO: 39) and (d) changes that were made to produce two exemplary Human Engineered™ sequences (SEQ ID NOs: 41 and 43). FIG. 9B shows the amino acid sequences of the two exemplary heavy chain Human Engineered™ sequences (SEQ ID NOs: 41 and 43), designated “low risk” and “low+moderate risk” as well as corresponding nucleic acid sequences (SEQ ID NOs: 40 and 42).

FIG. 10A shows (a) the risk line for the murine RX1 light chain (H-high risk, M=moderate risk, L=low risk), (b) the RX1 light chain amino acid sequence (SEQ ID NO: 5), (c) the amino acid sequence of the closest human consensus sequence, Kabat Vk3 consensus, aligned to RX1 (SEQ ID NO: 49) and (d) changes that were made to produce two exemplary Human Engineered™ sequences (SEQ ID NOs: 45 and 47). FIG. 10B shows the amino acid sequences of the two exemplary light chain Human Engineered™ sequences (SEQ ID NOs: 45 and 47), designated “low risk” and “low+moderate risk” as well as corresponding nucleic acid sequences (SEQ ID NOs: 44 and 46).

FIG. 11A shows (a) the risk line for the murine RX1 light chain (H=high risk, M=moderate risk, L=low risk), (b) the RX1 light chain amino acid sequence (SEQ ID NO: 5), (c) the amino acid sequence of the closest human consensus sequence, Kabat Vk3 consensus, aligned to RX1 (SEQ ID NO: 49) and (d) an alternate exemplary amino acid sequence in which positions 54-56 were not changed (i.e. remained the murine sequence) (SEQ ID NO: 48). FIG. 11B shows the amino acid sequences of two exemplary alternate light chain Human Engineered™ sequences (SEQ ID NOs: 48, 87), as well as corresponding nucleic acid sequences (SEQ ID NOs: 88 and 86).

FIG. 12A shows (a) the risk line for the murine RX1 light chain (H=high risk, M=moderate risk, L=low risk), (b) the RX1 light chain amino acid sequence (SEQ ID NO: 5), (c) the amino acid sequence of the closest human consensus germline sequence, Vk6 Subgroup 2-1-(1) A14, aligned to RX1 (SEQ ID NO: 50) and (d) changes that were made to produce two exemplary Human Engineered™ sequences (SEQ ID NOs: 51 and 53). FIG. 12B shows the amino acid sequences of the two exemplary light chain Human Engineered™ sequences (SEQ ID NOs: 51 and 53), designated “low risk” and “low+moderate risk” as well as the corresponding nucleic acid sequence (SEQ ID NO: 52).

FIGS. 13A and 24B show the alignment of murine RX1 heavy chain amino acid sequence (SEQ ID NO: 54) with various human consensus and human germline consensus sequences using the Kabat numbering system (amino acid numbering indicated in line designated “POS”) (SEQ ID NOs: 55-83). FIGS. 13C-13E show how the amino acid residues of antibodies 5H4, MC1 and MC3 correspond to the Kabat numbering system (SEQ ID NOs: 10 and 11; SEQ ID NOs: 12 and 13; SEQ ID NOs: 14 and 15, respectively).

FIG. 14 shows the anti-resorptive effects of Zometa in an animal model.

FIG. 15 shows the percent of animals in each group with detectable osteolysis.

FIG. 16 shows the mean osteolysis scores based on x-ray image analysis on the last day of the study.

FIG. 17 shows representative Faxitron x-ray images of tibias (tumor inoculation site) on the final day of the study. Arrows point to sites of osteolysis.

FIG. 18 shows the effect of RX1 on osteoclast activity.

FIG. 19 shows inhibition of osteclast activity by Zometa.

FIG. 20 shows the results of a pharmacokinetic study with RX1 in primates.

FIG. 21 shows the results of a pharmacokinetic study with RX1 in primates.

### DETAILED DESCRIPTION

Colony stimulating factor (CSF-1), also known as macrophage colony stimulating factor (M-CSF), has been found crucial for osteoclast formation. In addition, M-CSF has been shown to modulate the osteoclastic functions of mature osteoclasts, their migration and their survival in cooperation with other soluble factors and cell to cell interactions provided by osteoblasts and fibroblasts (Fixe and Praloran, Cytokine 10: 3-7, 1998; Martin et al., Critical Rev. in Eukaryotic Gene Expression 8: 107-23 (1998)).

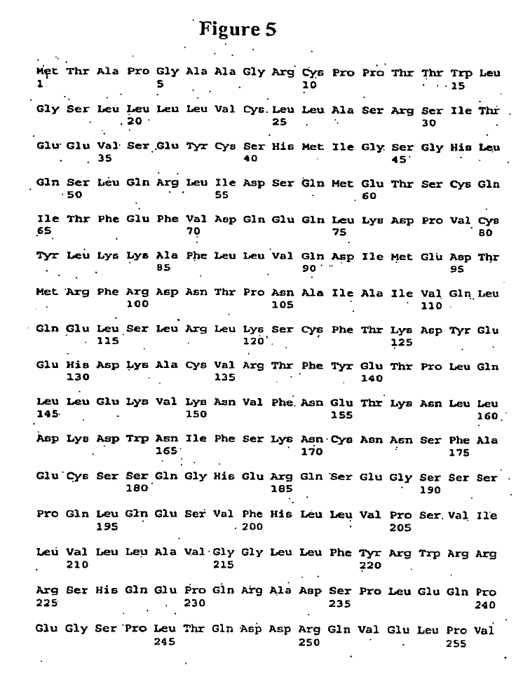

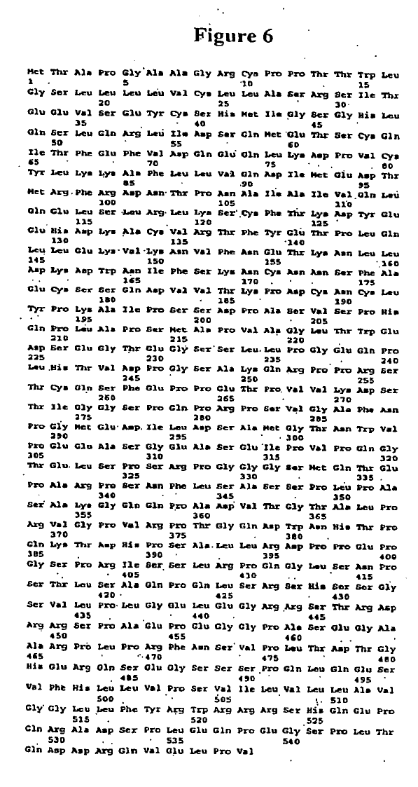

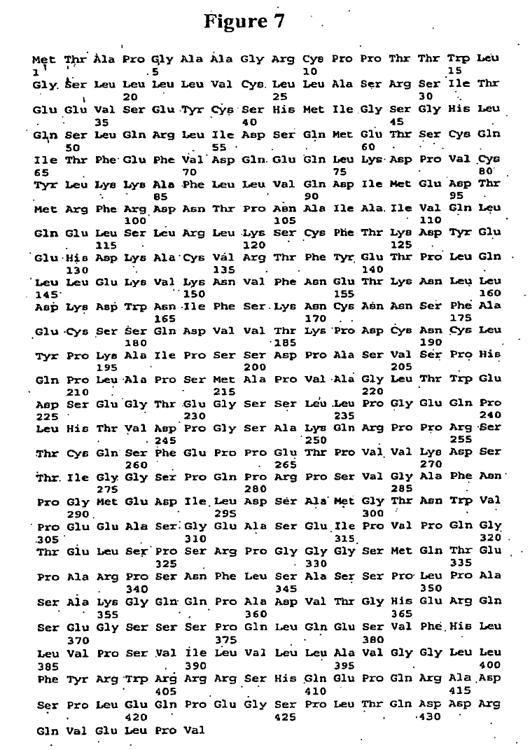

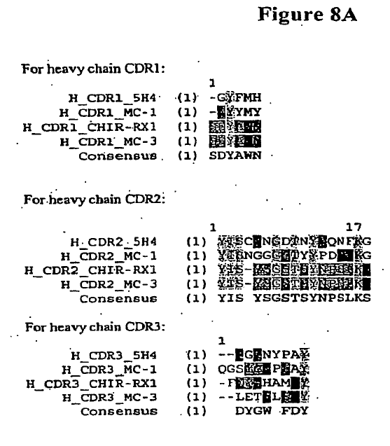

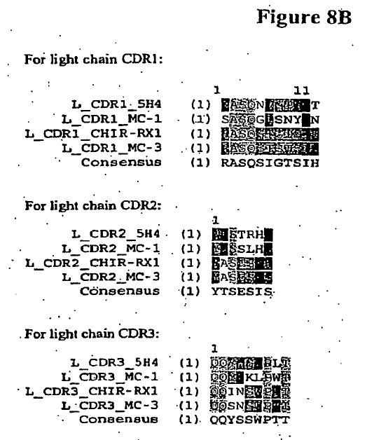

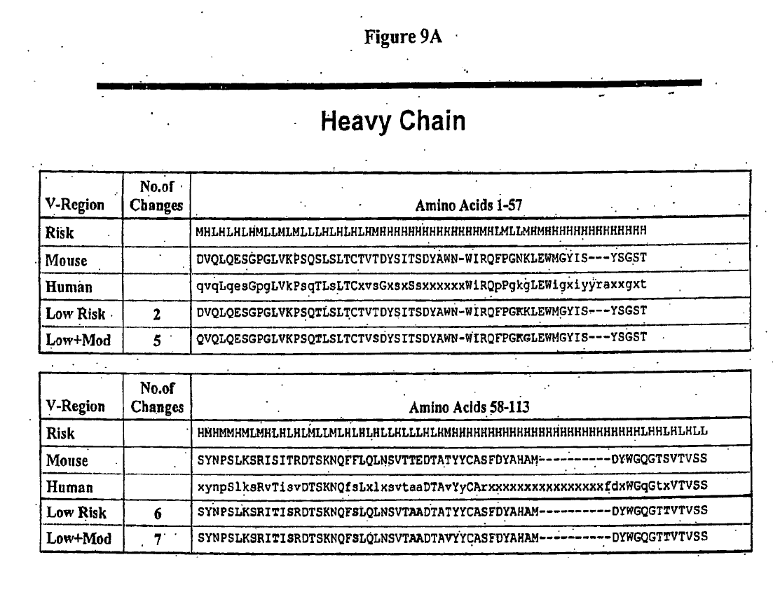

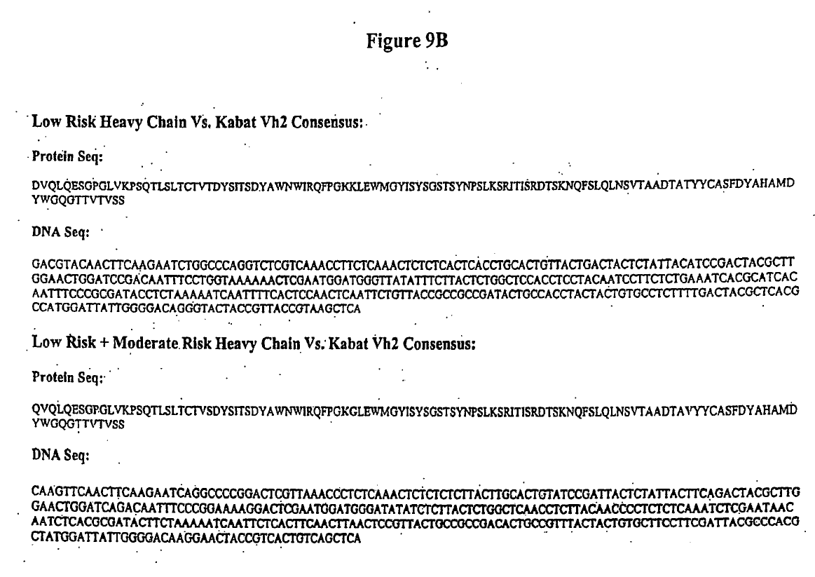

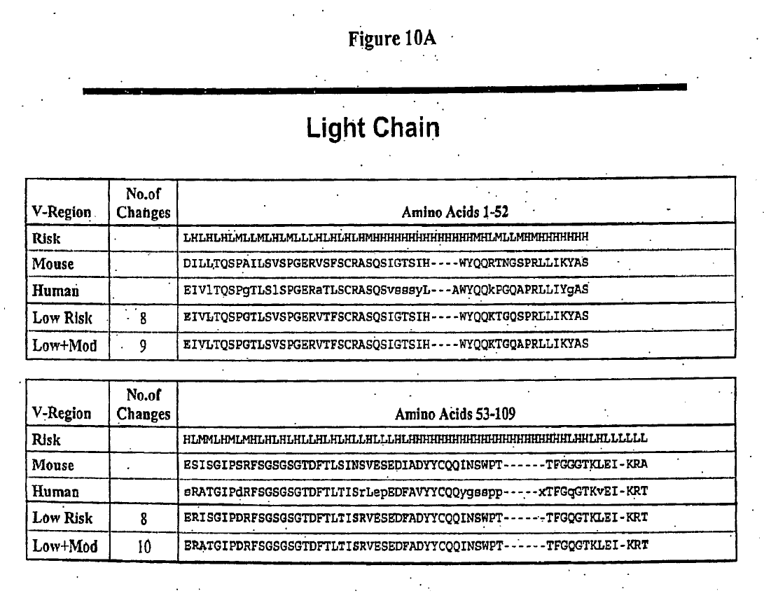

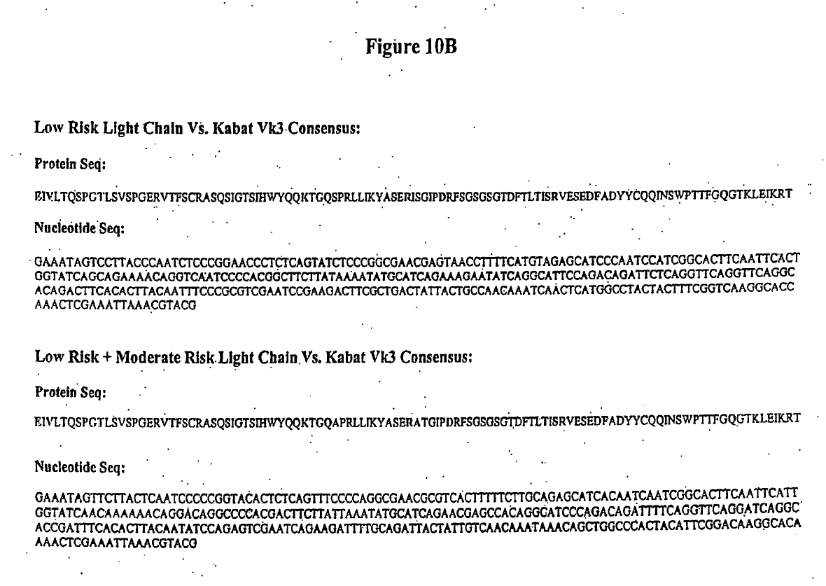

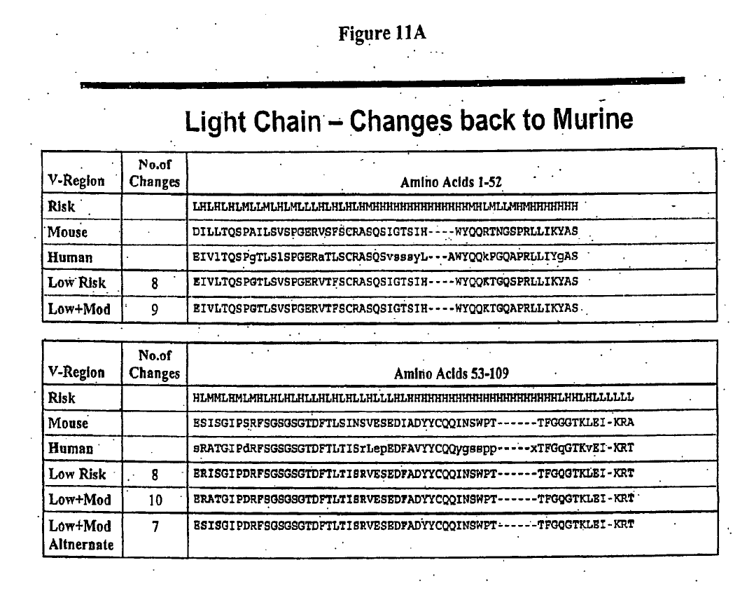

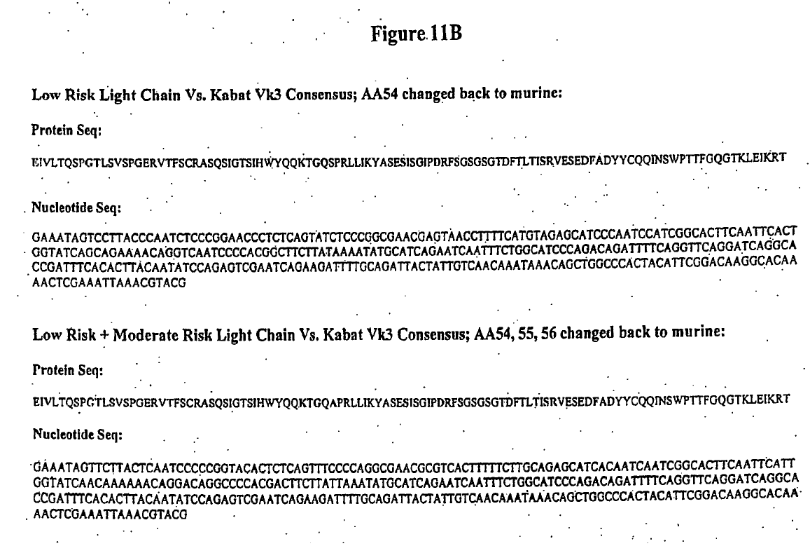

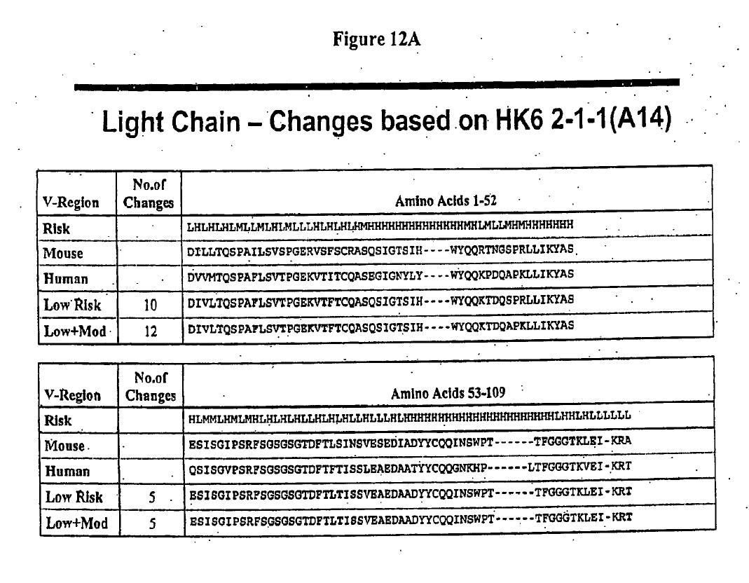

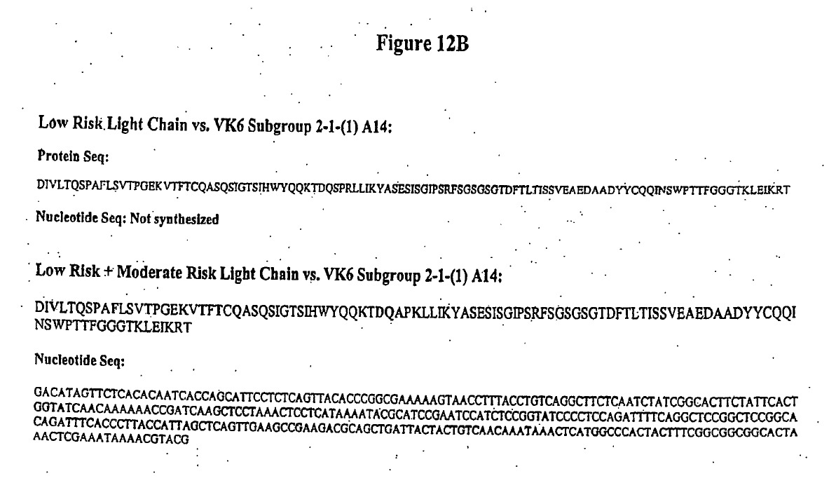

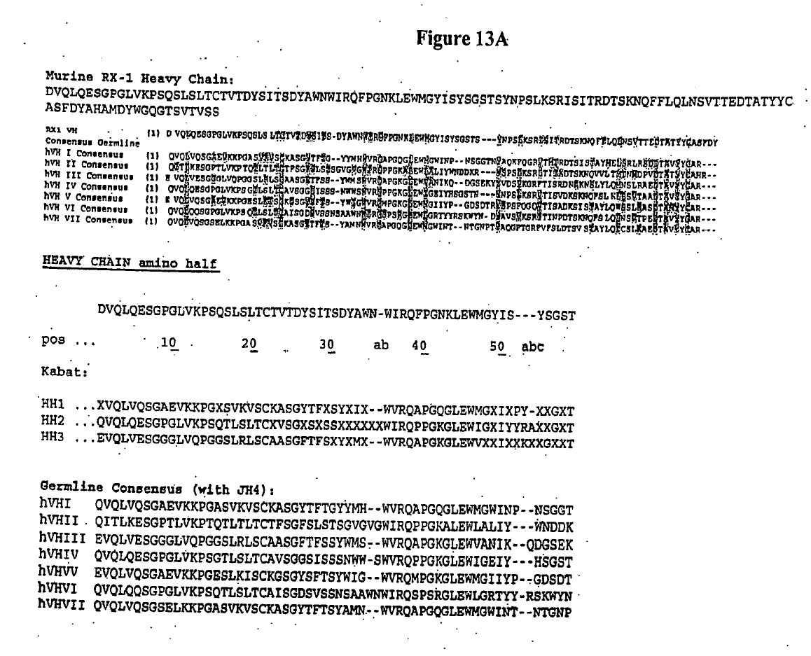

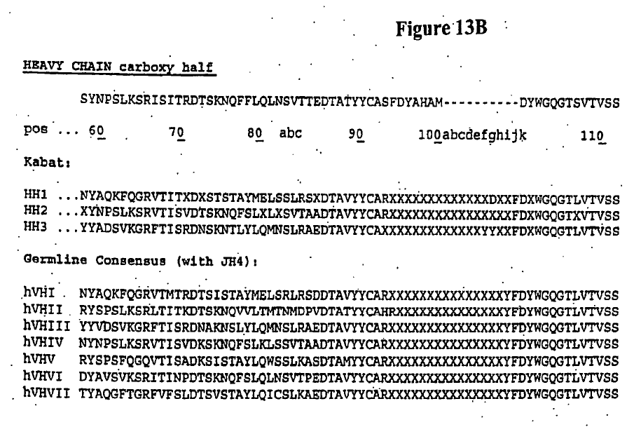

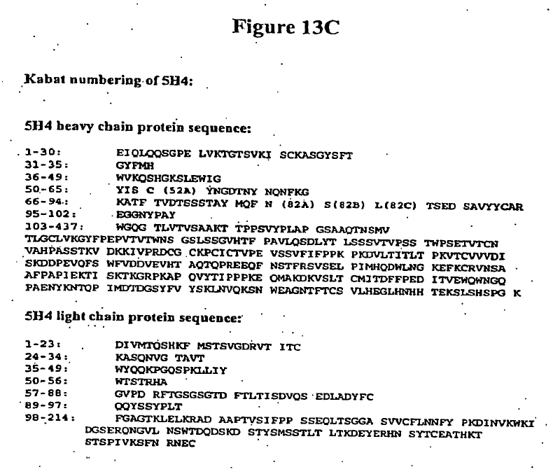

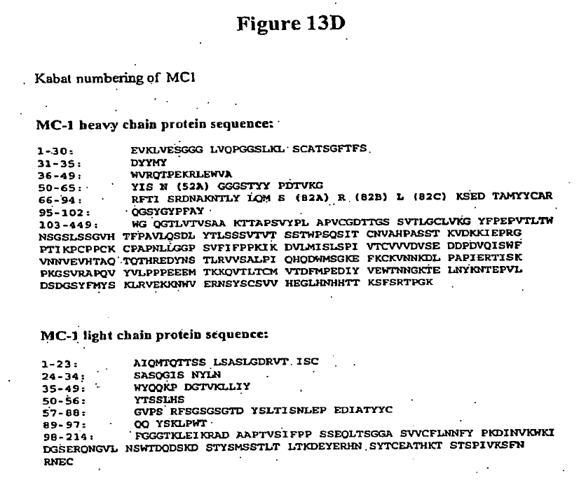

The full-length human M-CSF mRNA encodes a precursor protein of 554 amino acids. Through alternative mRNA splicing and differential post-translational proteolytic processing, M-CSF can either be secreted into the circulation as a glycoprotein or chondroitin sulfate containing proteoglycan or be expressed as a membrane spanning glycoprotein on the surface of M-CSF producing cells. The three-dimensional-structure of the bacterially expressed amino terminal 150 amino acids of human M-CSF, the minimal sequence required for full in vitro biological activity, indicates that this protein is a disulfide linked dimer with each monomer consisting of four alpha helical bundles and an anti-parallel beta sheet (Pandit et al., Science 258: 1358-62 (1992)). Three distinct M-CSF species are produced through alternative mRNA splicing. The three polypeptide precursors are M-CFSα of 256 amino acids, M-CSF, of 554 amino acids, and M-CSFγ of 438 amino acids. M-CSFα is a secreted protein that does not occur in a membrane-bound form. M-CSFα is expressed as an integral membrane protein that is slowly released by proteolytic cleavage. M-CSFβ is cleaved at amino acids 191-197 of the sequence set out in FIG. 5. The membrane-bound form of M-CSF can interact with receptors on nearby cells and therefore mediates specific cell-to-cell contacts. The term “M-CSF” may also include amino acids 36-438 of FIG. 7.

Various forms of M-CSF function by binding to its receptor M-CSFR on target cells. M-CSFR is a membrane spanning molecule with five extracellular immunoglobulin-like domains, a transmembrane domain and an intracellular interrupted Src related tyrosine kinase domain. M-CSFR is encoded by the c-fms proto-oncogene. Binding of M-CSF to the extracellular domain of M-CSFR leads to dimerization of the receptor, which activates the cytoplasmic kinase domain, leading to autophosphorylation and phosphorylation of other cellular proteins (Hamilton J. A., J Leukoc Biol., 62(2):145-55, 1997; Hamilton J, A., Immuno Today., 18(7): 313-7, 1997).

Phosphorylated cellular proteins induce a cascade of biochemical events leading to cellular responses: mitosis, secretion of cytokines, membrane ruffling, and regulation of transcription of its own receptor (Fixe and Praloran, Cytokine 10: 32-37, 1998).

“Tumor”, as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, carcinoma; lymphoma, blastoma, sarcoma and leukemia. More particular examples of such cancers include breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma; kidney cancer, liver cancer, vulval cancer, thyroid cancer, hepatic carcinoma and various types of head and neck cancer.

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. The “pathology” of cancer includes all phenomena that compromise the well being of the patient. This includes, without limitation, abnormal or uncontrollable cell growth; metastasis, interference with the normal functioning of neighboring cells, release of cytokines or other secretory products at abnormal levels, suppression or aggravation of inflammatory or immunological response, etc.

“Mammal” for purposes of treatment refers to any animal classified as a mammal, including humans, domestic and farm animals, and zoo, sports, or pet animals, such as dogs, horses, cats, cows, etc. Preferably, the mammal is human.

As used herein, the phrase “metastatic cancer” is defined as cancers that have potential to spread to other areas of the body, particularly bone. A variety of cancers can metastasize to the bone, but the most common metastasizing cancers are breast, lung, renal, multiple myeloma, thyroid and prostate. By way of example, other cancers that have the potential to metastasize to bone include but are not limited to adenocarcinoma, blood cell malignancies, including leukemia and lymphoma; head and neck cancers; gastrointestinal cancers, including esophageal cancer, stomach cancer, colon cancer, intestinal cancer, colorectal cancer, rectal cancer, pancreatic cancer, liver cancer, cancer of the bile duct or gall bladder; malignancies of the female genital tract, including ovarian carcinoma, uterine endometrial cancers, vaginal cancer, and cervical cancer; bladder cancer; brain cancer, including neuroblastoma; sarcoma, osteosarcoma; and skin cancer, including malignant melanoma and squamous cell cancer. The present invention especially contemplates prevention and treatment of tumor-induced osteolytic lesions in bone.

As used herein, the phrase “therapeutically effective amount” refers to is meant to refer to an amount of therapeutic or prophylactic M-CSF antagonist, such as M-CSF antibody, that would be appropriate for an embodiment of the present invention, that will elicit the desired therapeutic or prophylactic effect or response when administered in accordance with the desired treatment regimen.

Human “M-CSF” as used herein refers to a human polypeptide having substantially the same amino acid sequence as the mature human M-CSFα, M-CSFβ, or M-CSFγ polypeptides described in Kawasaki et al., Science 230:291 (1985), Cerretti et al., Molecular Immunology, 25:761 (1988), or Ladner et al., EMBO Journal 6:2693 (1987), each of which are incorporated herein by reference. Such terminology reflects the understanding that the three mature M-CSs have different amino acid sequences, as described above, and that the active form of M-CSF is a disulfide bonded dimer; thus, when the term “M-CSF” refers to the biologically active form, the dimeric form is intended. “M-CSF dimer” refers to two M-CSF polypeptide monomers that have dimerized and includes both homodimers (consisting of two of the same type of M-CSF monomer) and heterodimers (consisting of two different monomers). M-CSF monomers may be converted to M-CSF dimers in vitro as described in U.S. Pat. No. 4,929,700, which is incorporated herein by reference.

I. Antagonists

As used herein, the term “antagonist” generally refers to the property of a molecule, compound or other agent to, for example, interfere with the binding of one molecule with another molecule or the stimulation of one cell by another cell either through steric hindrance, conformational alterations or other biochemical mechanisms. In one regard, the term antagonist relates to the property of an agent to prevent the binding of a receptor to its ligand, e.g., the binding of M-CSF with M-CSFR, thereby inhibiting the signal transduction pathway triggered by M-CSF. The term antagonist is not limited by any specific action mechanism, but, rather, refers generally to the functional property presently defined. Antagonists of the present invention include, but are not limited to: M-CSF antibodies and fragments and muteins and modifications thereof, soluble M-CSF and fragments and muteins and modifications thereof, M-CSFR antibodies and fragments and muteins and modifications thereof, soluble M-CSFR and fragments and muteins and modifications thereof, and peptides as well as other chemical compounds and molecules that bind to M-CSF or M-CSFR and nucleic acid molecules such as antisense or RNAi compounds that inhibit expression of M-CSF and M-CSFR. Any of the antagonists of the present invention can be administered in any manner known in the art. For example, M-CSF muteins, M-CSFR muteins or antibody fragments that bind to M-CSF or M-CSFR can be administered via gene therapy.

M-CSF antagonists of the present invention include, where applicable, functional equivalents. For example, molecules may differ in length, structure, components, etc., but may still retain one or more of the defined functions. More particularly, functional equivalents of the antibodies, antibody fragments or peptides of the present invention may include mimetic compounds, i.e., constructs designed to mimic the proper configuration and/or orientation for antigen binding.

Preferred M-CSF antagonists may optionally be modified by addition of side groups, etc., e.g., by amino terminal acylation, carboxy terminal amidation or by coupling of additional groups to amino acid side chains. Antagonists may also comprise one or more conservative amino acid substitutions. By “conservative amino acid substitutions” is meant those changes in amino acid sequence that preserve the general charge, hydrophobicity/hydrophilicity and/or steric bulk of the amino acid substituted. For example, substitutions between the following groups are conservative: Gly/Ala, Val/Ile/Leu, Asp/Glu, Lys/Arg, Asn/Gln, Ser/Cys/Thr, and Phe/Trp/Tyr. Such modifications will not substantially diminish the efficacy of the M-CSF antagonists and may impart such desired properties as, for example, increased in vivo half life or decreased toxicity.

The invention is also intended to include polypeptides bearing modifications other than the insertion, deletion, or substitution of amino acid residues. By way of example, the modifications may be covalent in nature, and include for example, chemical bonding with polymers, lipids, other organic, and inorganic moieties. Such derivatives may be prepared to increase circulating half-life of a polypeptide, or may be designed to improve targeting capacity for the polypeptide to desired cells, tissues, or organs. Similarly, the invention further embraces M-CSF or M-CSFR polypeptides that have been covalently modified to include one or more water soluble polymer attachments such as polyethylene glycol, polyoxyethylene glycol, or polypropylene glycol.

A. M-CSF Antibodies

The term “antibody” is used in the broadest-sense and includes fully assembled antibodies, monoclonal antibodies, polyclonal antibodies, multispecific antibodies (e.g., bispecific antibodies), antibody fragments that can bind antigen (e.g., Fab′, F′(ab)2, Fv, single chain antibodies, diabodies), and recombinant peptides comprising the forgoing as long as they exhibit the desired biological activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that are typically include different antibodies directed against different determinants (epitopes), each monoclonal antibody is directed against a single determinant on the antigen. In addition to their specificity, the monoclonal antibodies are advantageous in that they are synthesized by the homogeneous culture, uncontaminated by other immunoglobulins with different specificities and characteristics.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 [1975], or may be made by recombinant DNA methods (see, e.g., U.S. Pat. No. 4,816,567). The “monoclonal antibodies” may also be isolated from phage antibody libraries using the techniques described in Clackson et al., Nature, 352:624628[1991] end Marks et al., J. Mol. Biol., 222.1581-597 (1991), for example.

Depending on the amino acid sequence of the constant domain of their heavy chains, immunoglobulins can be assigned to different classes. There are five major classes, IgA, IgD, IgE, IgG and IgM, and several of these may be further divided into subclasses or isotypes, e.g. IgG1, IgG2, IgG3, IgG4, IgA1 and IgA2. The heavy-chain constant domains that correspond to the different classes of immunoglobulins are called alpha, delta, epsilon, gamma- and mu respectively. The subunit structures and three-dimensional configurations of different classes of immunoglobulins are well known. Different isotypes have different effector functions; for example, IgG1 and IgG3 isotypes have ADCC activity.

“Antibody fragments” comprise a portion of an intact full length antibody, preferably the antigen binding or variable region of the intact antibody. Examples of antibody fragments include Fab, Fab′, F(ab′)2, and Fv fragments; diabodies; linear antibodies (Zapata et al., Protein Eng., 8(10):1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize 35 readily. Pepsin treatment yields an F(ab′)2 fragment that has two “Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains that enables the Fv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

The term “hypervariable” region refers to the amino acid residues of an antibody which are responsible for antigen-binding. The hypervariable region comprises amino acid residues from a complementarity determining region or CDR [i.e., residues 24-34 (L1), 50-56 (L2) and 89-97 (L3) in the light chain variable domain and 31-35 (H1), 50-65 (H2) and 95-102 (H3) in the heavy chain variable domain as described by Kabat-et al., Sequences of Proteins of Immunological Interest, 5^{th} Ed. Public Health Service, National Institutes of Health, Bethesda, Md. (1991)] and/or those residues from a hypervariable loop (i.e., residues 26-32 (L1), 50-52 (L2) and 91-96 (L3) in the light chain variable domain and 26-32 (H1), 53-55 (H2) and 96-101 (H3) in the heavy chain variable domain as described by [Chothia et al., J. Mol. Biol. 196: 901-917 (1987)].

“Framework” or FR residues are those variable domain residues other than the hypervariable region residues.

The term “diabodies” refers to small antibody fragments with two antigen-binding sites, which fragments comprise a heavy-chain variable domain (VH) connected to a light-chain variable domain (VL) in the same polypeptide chain (VH VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and 30 Hollinger et al., Proc. Natl. Acad. Sci. USA, 90:6444-6448 (1993).

In some embodiments, it may be desirable to generate multispecific (e.g. bispecific) monoclonal antibody including monoclonal, human, humanized, Human Engineered™ or variant anti-M-CSF antibodies having binding specificities for at least two different epitopes. Exemplary bispecific antibodies may bind to two different epitopes of M-CSF. Alternatively, an anti-M-CSF arm may be combined with an arm which binds to a triggering molecule on a leukocyte such as a T-cell receptor molecule (e.g., CD2 or CD3), or Fc receptors for IgG (FcγR), such as FcγRI (CD64), FcγRII (CD32) and FcγRIII (CD16) so as to focus cellular defense mechanisms to the M-CSF-expressing cell. Bispecific antibodies may also be used to localize cytotoxic agents to cells which express M-CSF. These antibodies possess an M-CSF-binding arm and an arm which binds the cytotoxic agent (e.g., saporin, anti-interferon-60, vinca alkaloid, ricin A chain, methotrexate or radioactive isotope hapten). Bispecific antibodies can be prepared as full length antibodies or antibody fragments (e.g., F(ab′).sub.2 bispecific antibodies).

According to another approach for making bispecific antibodies, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. See WO96/27011 published Sep. 6, 1996.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes. In yet a further embodiment, Fab′-SH fragments directly recovered from E. coli can be chemically coupled in vitro to form bispecific antibodies. (Shalaby et al., J. Exp. Med. 175:217-225 (1992))

Shalaby et al., J. Exp. Med. 175:217-225(1992) describe the production of a fully humanized bispecific antibody F(ab′)₂ molecule. Each Fab′ fragment was separately secreted from E. coli and subjected to directed chemical coupling in vitro to form the bispecfic antibody. The bispecific antibody thus formed was able to bind to cells overexpressing the HER2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. (Kostelny et al., J. Immunol. 148(5):1547-1553 (1992)) The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments.

The fragments comprise a heavy chain variable region (V_{H}) connected to a light-chain variable region (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol. 152: 5368 (1994).

Alternatively, the bispecific antibody may be a “linear antibody” produced as described in Zapata et al. Protein Eng. 8(10):1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

Antibodies with more than two valencies are also contemplated. For, example, trispecific antibodies can be prepared. (Tutt et al., J. Immunol. 147:60 (1991))

In certain embodiments, the monoclonal, human, humanized, Human Engineered™ or variant anti-M-CSF antibody is an antibody fragment, such as an RX1, 5H4, MC1, or MC3 antibody fragment. Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science 229:81 (1985)). However, these fragments can now be produced directly by recombinant host cells. Better et al., Science 240: 1041-1043 (1988) disclose secretion of functional antibody fragments from bacteria (see, e.g., Better et al., Skerra et al., Science 240: 1038-1041 (1988)). For example, Fab′-SH fragments can be directly recovered from E. coli and chemically coupled to form F(ab′)₂ fragments (Carter et al., Bio/Technology 10:163-167 (1992)). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture. Other techniques for the production of antibody fragments will be apparent to the skilled practitioner.

An “isolated” antibody is one that has been identified and separated and for recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

For a detailed description of the structure and generation of antibodies, see Roth, D. B., and Craig, N. L., Cell, 94:411-414 (1998), and U.S. Pat. No. 6,255,458, herein incorporated by reference in its entirety. Briefly, the process for generating DNA encoding the heavy and light chain immunoglobulin genes occurs primarily in developing B-cells. Prior to the rearranging and joining of various immunoglobulin gene segments, the V, D, J and constant (C) gene segments are found generally in relatively close proximity on a single chromosome. During B-cell-differentiation, one of each of the appropriate family members of the V, D, J (or only V and J in the case of light chain genes) gene segments are recombined to form functionally rearranged heavy and light immunoglobulin genes. This gene segment rearrangement process appears to be sequential. First, heavy chain D-to-J joints are made, followed by heavy chain V-to-DJ joints and light chain V-to-J joints.

The recombination of variable region gene segments to form functional heavy and light chain variable regions is mediated by recombination signal sequences (RSS's) that flank recombinationally competent V, D and J segments. RSS's necessary and sufficient to direct recombination, comprise a dyad-symmetric heptamer, an AT-rich nonamer and an intervening spacer region of either 12 or 23 base pairs. These signals are conserved among the different loci and species that carry out D-J (or V-J) recombination and are functionally interchangeable. See Oettinger, et al. (1990), Science, 248, 1517-1523 and references cited therein. The heptamer comprises the sequence CACAGTG or its analogue followed by a spacer of unconserved sequence and then a nonamer having the sequence ACAAAAACC or its analogue. These sequences are found on the J, or downstream side, of each V and D gene segment. Immediately preceding the germline D and 3 segments are again two recombination signal sequences, first the nonamer and then the heptamer again separated by an unconserved sequence. The heptameric and nonameric sequences following a V_{L}, V_{H} or D segment are complementary to those preceding the J_{L}, D or J_{H} segments with which they recombine. The spacers between the heptameric and nonameric sequences are either 12-base pairs long or between 22 and 24 base pairs long.

In addition to the rearrangement of V, D and J segments, further diversity is generated in the primary repertoire of immunoglobulin heavy and light chain by way of variable recombination at the locations where the V and J segments in the light chain are joined and where the D and J segments of the heavy chain are joined. Such variation in the light chain typically occurs within the last codon of the V gene segment and the first codon of the J segment. Similar imprecision in joining occurs on the heavy chain chromosome between the D and J_{H} segments and may extend over as many as 10 nucleotides. Furthermore, several nucleotides may be inserted between the D and J_{H} and between the V_{H} and D gene segments which are not encoded by genomic DNA. The addition of these nucleotides is known as N-region diversity.

The net effect of such rearrangements in the variable region gene segments and the variable recombination which may occur during such joining is the production of a primary antibody repertoire.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen binding site on the surface of the VH VI dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them.

By “neutralizing antibody” is meant an antibody molecule that is able to eliminate or significantly reduce an effecter function of a target antigen to which is binds. Accordingly, a “neutralizing” anti-target antibody is capable of eliminating or significantly reducing an effecter function, such as enzyme activity, ligand binding, or intracellular signaling.

As provided herein, the compositions for and methods of treating cancer metastasis and/or bone loss associated with cancer metastasis may utilize one or more antibody used singularly or in combination with other therapeutics to achieve the desired effects. Antibodies according to the present invention may be isolated from an animal producing the antibody as a result of either direct contact with an environmental antigen or immunization with the antigen. Alternatively, antibodies may be produced by recombinant DNA methodology using one of the antibody expression systems' well known in the art (See, e.g., Harlow and Lane, Antibodies: A Laboratory Manual, Cold Spring Harbor Laboratory (1988)). Such antibodies may include recombinant IGs, chimeric fusion-proteins having immunoglobulin derived sequences or “Human Engineered™” antibodies that may all be used for the treatment of cancer metastasis and/or bone loss associated with cancer metastasis according to the present invention. In addition to intact, full-length molecules, the term “antibody” also refers to fragments thereof (such as, e.g., scFv, Fv, Fd, Fab, Fab′ and F(ab)′2 fragments) or multimers or aggregates of intact molecules and/or fragments that bind to M-CSF (or M-CSFR). These antibody fragments bind antigen and may be derivatized to exhibit structural features that facilitate clearance and uptake, e.g., by incorporation of galactose residues.

In one embodiment of the present invention, M-CSF monoclonal antibodies may be prepared essentially as described in Halenbeck et al. U.S. Pat. No. 5,491,065 (1997), incorporated herein by reference. Exemplary M-CSF monoclonal antibodies include those that bind to an apparent conformational epitope associated with recombinant or native dimeric M-CSF with concomitant neutralization of biological activity. These antibodies are substantially unreactive with biologically inactive forms of M-CSF including monomeric and chemically derivatized dimeric M-CSF.

In other embodiments of the present invention, Human Engineered anti-M-CSF monoclonal antibodies are provided. The phrase “Human Engineered antibody” refers to an antibody derived from a non-human antibody, typically a mouse monoclonal antibody. Alternatively, a Human Engineered antibody may be derived from a chimeric antibody that retains or substantially retains the antigen binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

The phrase “complementarity determining region” or the term “CDR” refers to amino acid sequences which together define the binding affinity and specificity of the natural Fv region of a native immunoglobulin binding site (See, e.g., Chothia et al., J. Mol. Biol. 196:901 917 (1987); Kabat et al., U.S. Dept. of Health and Human Services NIH Publication No. 91 3242 (1991)). The phrase “constant region” refers to the portion of the antibody molecule that confers effector functions. In the present invention, mouse constant regions are preferably substituted by human constant regions. The constant regions of the subject antibodies are derived from human immunoglobulins. The heavy chain constant region can be selected from any of the five isotypes: alpha, delta, epsilon, gamma or mu.

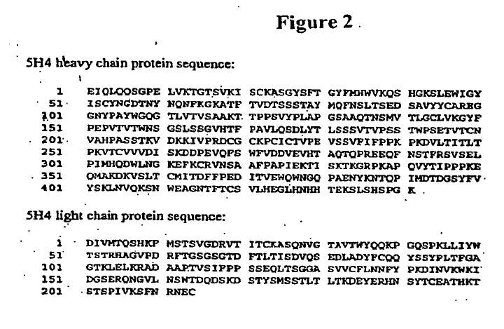

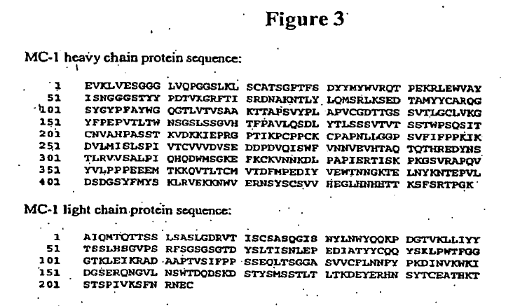

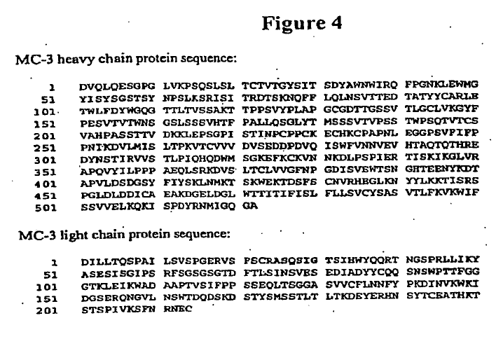

The antibodies of the present invention are said to be immunospecific or specifically binding if they bind to antigen with a Kₐ of greater than or equal to about 10⁶M⁻¹ preferably greater than or equal to about 10⁷M⁻¹, more preferably greater than or equal to about 10⁸M⁻¹, and most preferably greater than or equal to about 10⁹M⁻¹, 10¹⁰M⁻¹, 10¹¹M⁻¹ or 10¹²M⁻¹. The anti-M-CSF antibodies may bind to different naturally occurring forms of M-CSF, including those expressed by the host's/subject's tissues as well as that expressed by the tumor. The monoclonal antibodies disclosed herein, such as RX1, 5H4, MC1, or MC3 antibody, have affinity for M-CSF and are characterized by a dissociation equilibrium constant (Kd) of at least 10⁻⁴ M, preferably at least about 10⁻⁷ M to about 10⁻⁸ M, more preferably at least about 10-⁸M, 10-¹⁰M, 10-¹¹ M or 10-¹²M. Such affinities may be readily determined using conventional techniques, such as by equilibrium dialysis; by using the BIAcore 2000 instrument, using general procedures outlined by the manufacturer; by radioimmunoassay using ¹²⁵I labeled M-CSF; or by another method known to the skilled artisan. The affinity data may be analyzed, for example, by the method of Scatchard et al., Ann N.Y. Acad. Sci., 51:660 (1949). Thus, it will be apparent that preferred M-CSF antibodies will exhibit a high degree of specificity for M-CSF and will bind with substantially lower affinity to other molecules. Preferred antibodies bind M-CSF with a similar affinity as murine RX1 of FIG. 4 binds to M-CSF, exhibit low immunogenicity, and inhibit metastasis of cancer cells when tested in metastatic disease animal models. Other exemplary antibodies bind M-CSF with a similar affinity as murine 5H4, MC1 or MC3 of FIG. 2, 3 or 4, respectively, binds to M-CSF.

The antigen to be used for production of antibodies may be, e.g., intact M-CSF or a fragment of M-CSF that retains the desired epitope, optionally fused to another polypeptide that allows the epitope to be displayed in its native conformation. Alternatively, cells expressing M-CSF at their cell surface can be used to generate antibodies. Such cells can be transformed to express M-CSF or may be other naturally occurring cells that express M-CSF. Other forms of M-CSF useful for generating antibodies will be apparent to those skilled in the art.

i. Polyclonal Antibodies

Polyclonal antibodies are preferably raised in animals by multiple subcutaneous (sc) or intraperitoneal (ip) injections of the relevant antigen and an adjuvant. An improved antibody response may be obtained by conjugating the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride or other agents known in the art.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. At 7-14 days post-booster injection, the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

ii Monoclonal Antibodies

Monoclonal antibodies may be made using the hybridoma method first described by Kohler et al., Nature, 256:495 (1975), or may be made by recombinant DNA methods.

In the hybridoma method, a mouse or other appropriate host animal, such as a hamster or macaque monkey, is immunized as herein described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)). Exemplary murine myeloma lines include those derived from MOP-21 and M.C.-11 mouse tumors available from the Salk Institute Cell Distribution Center, San Diego, Calif. USA, and SP-2 or X63-Ag8-653 cells available from the American Type Culture Collection, Rockville, Md. USA.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by Scatchard analysis (Munson et al., Anal. Biochem., 107:220 (1980)).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

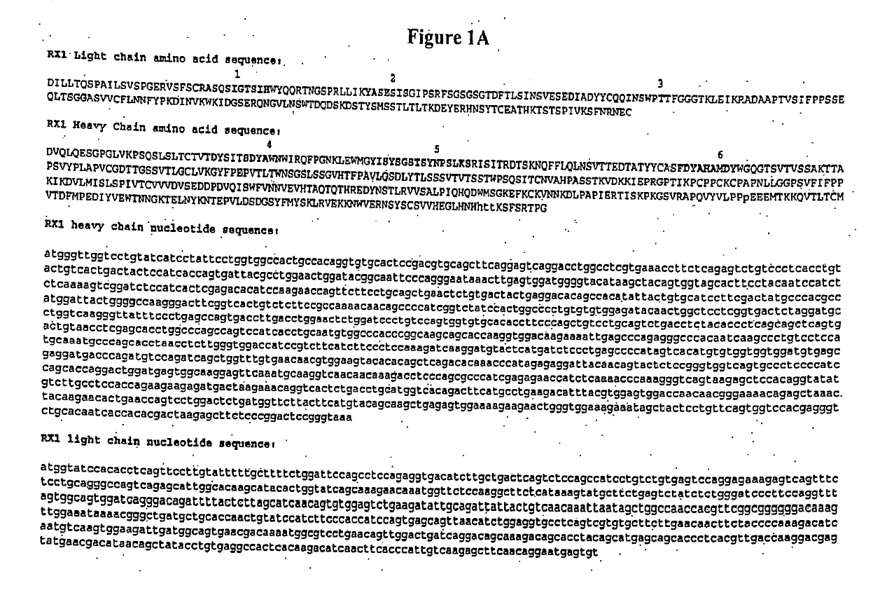

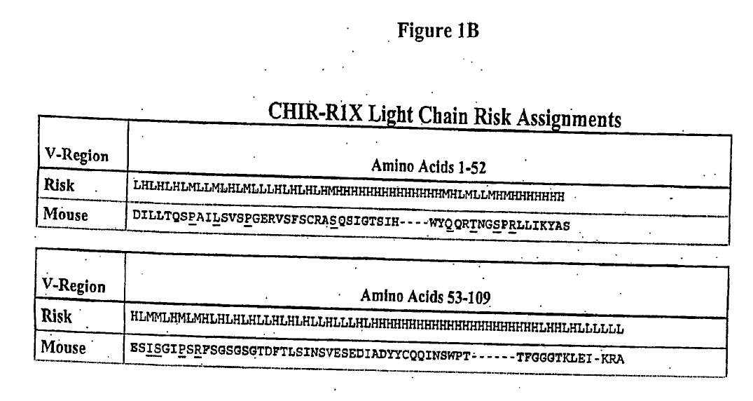

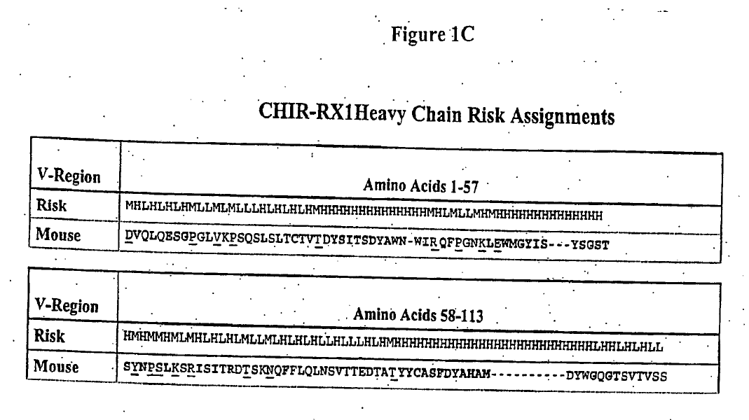

The antibodies of the present invention are said to be immunospecific or specifically binding if they bind to antigen with a Kₐ of greater than or equal to about 10⁶M⁻¹ preferably greater than or equal to about 10⁷M⁻¹, more preferably greater than or equal to about 10⁸M⁻¹, and most preferably greater than or equal to about 10⁹M⁻¹, 10¹⁰M⁻¹, 10¹¹ M⁻¹ or 10²M⁻¹. The anti-M-CSF antibodies may bind to different naturally occurring forms of M-CSF, including those expressed by the host's/subject's tissues as well as that expressed by the tumor. The monoclonal antibodies disclosed herein, such as RX1, 3H4, MC1, or MC3 antibody, have affinity for M-CSF and are characterized by a dissociation equilibrium constant (Kd) of at least 10⁻⁴ M, preferably at least about 10⁻⁷ M to about 10⁻⁸ M, more preferably at least about 10-⁸ M, 10-¹⁰ M, 10-¹¹ M or 10-¹²M. Such affinities may be readily determined using conventional techniques, such as by equilibrium dialysis; by using the BIAcore 2000 instrument, using general procedures outlined by the manufacturer; by radioimmunoassay using ¹²⁵I labeled M-CSF; or by another method known to the skilled artisan. The affinity data may be analyzed, for example, by the method of Scatchard et al., Ann N.Y. Acad. Sci., 51:660 (1949). Thus, it will be apparent that preferred M-CSF antibodies will exhibit a high degree of specificity for M-CSF and will bind with substantially lower affinity to other molecules. Preferred antibodies bind M-CSF with a similar affinity as murine RX1 of FIG. 1 binds to M-CSF, exhibit low immunogenicity, and inhibit metastasis of cancer cells when tested in metastatic disease animal models. Other exemplary antibodies bind M-CSF with a similar affinity as murine 51-4, MC1 or MC3 of FIG. 2, 3 or 4, respectively, binds to M-CSF.

Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE 1

Original Exemplary Preferred
Residue Substitutions

| | |
|---|---|
| Ala (A) val; leu; ile | Arg (R) lys; gln; asn lys |
| val | |
| Asn (N) gln; his; asp, | Asp (D) glu; asn glu |
| lys; gln arg | |
| Cys (C) ser; ala ser | Gln (Q) asn; gln asn |
| Glu (E) asp; gln asp | Gly (G) ala |
| His (H) asn; gln; | Ile (I) leu; val; met; ala; |
| lys; arg | leu phe; norleucine |

Leu (L) norleucine; ile; val; ile met; ala; phe

| | |
|---|---|
| Lys (K) arg; gln; | Met (M) leu; phe; ile leu |
| asn arg | |
| Phe (F) leu; val; | Pro (P) ala |
| ile; ala; tyr | |
| Ser (S) thr | Thr (T) ser ser |
| Trp (W) tyr; phe tyr | Tyr (Y) trp; phe; thr; ser |
| | phe |

Val (V) ile; leu; met; phe; leu ala; norleucine

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met; ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions involve replacing a member of one of these classes with a member of another class.

Any cysteine residue not involved in maintaining the proper conformation of the humanized or variant antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

B. M-CSF Muteins

The invention further provides M-CSF muteins that may be used as MCSF antagonists according to the methods of the invention.

“Fragment” as used herein means a portion of the intact native molecule; for example, a fragment polypeptide is a fragment of the native polypeptide in which one or more amino acids from either the N-terminal or C-terminal have been deleted.

“Mutein” as used herein with respect to polypeptides means a variant of the intact native molecule or a variant of a fragment of the native molecule, in which one or more amino acids have been substituted, inserted or deleted. Such substitutions, insertions or deletions can be at the N-terminus, C-terminus or internal to the molecule. Thus the term “muteins” includes within its scope fragments of the native molecule. Insertional muteins include fusions at the N- or C-terminus, e.g. fusion to the Fc portion of an immunoglobulin to increase half-life

Preferred muteins according to the invention exhibit at least about 65%, 70%. 75%, 80%, 85%, 90%, 95%, 97% or more sequence identity (homology) to the native polypeptide, as determined by the Smith-Waterman homology search algorithm (Meth. Mol. Biol. 70:173-187 (1997)) as implemented in the MSPRCH program (Oxford Molecular) using an affine gap search with the following search parameters: gap open penalty of 12, and gap extension penalty of 1. Other well-known and routinely used homology/identity scanning algorithm programs include Pearson and Lipman, PNAS USA, 85:2444-2448 (1988); Lipman and Pearson, Science, 222:1435 (1985); Devereaux et al., Nuc. Acids Res., 12:387-395 (1984); or the BLASTP, BLASTN or BLASTX algorithms of Altschul, et al., Mol. Biol., 215:403-410 (1990). Computerized programs using these algorithms are also available and include, but are not limited to: GAP, BESTFIT, BLAST, FASTA and TFASTA, which are commercially available from the Genetics Computing Group (GCG) package, Version 8, Madison Wis., USA; and CLUSTAL in the PC/Gene program by Intellegenetics, Mountain View Calif. Preferably, the percentage of sequence identity is determined by using the default parameters determined by the program.

“Modification” as used herein means any modification of the native polypeptide, fragment or mutein, such as glycosylation, phosphorylation, polymer conjugation (such as with polyethylene glycol), or other addition of foreign moieties, so long as the desired activity (agonist or antagonist) is retained.

U.S. Pat. No. 6,025,146, and Koths, Mol. Reprod. Dev. 1997 January;46(1):31-38 both of which are incorporated herein by reference in their entirety, describe the crystallization of M-CSF alone and M-CSF complexed to MCSF-R, and characterize the three-dimensional structure of M-CSF as well as residues involved in receptor-binding. U.S. Pat. No. 6,025,146 also describes methods for selecting candidate amino acid substitutions in M-CSF, based on structural information. The overall topology of this form of M-CSF is that of an antiparallel four alpha-helical bundle, in which the helices run up-up-down-down, unlike the more commonly observed up-down-up-down connectivity of most four helical bundles. A long crossover connection links helix A to helix B and a similar connection is found between helices C and D. In the disulfide-linked dimeric form, the bundles are linked end-to-end, forming an extremely flat, elongated structure (approximate dimensions 85×35×25). The re are three intramolecular disulfide bonds in each monomer (Cys7-Cys90, Cys48-Cys139, Cys102-Cys146) all of which are at the distal end of the molecule. One interchain disulfide bond (Cys31-Cys31) is located at the dimer interface with the noncrystallographic two-fold symmetry axis passing through it as shown in FIG. 2. Mutation experiments indicate that all of the cysteine residues in this form of M-CSF may be necessary for full biological activity. The structure described herein suggests that their role is primarily structural rather than being related to receptor recognition. U.S. Pat. No. 6,025,146 provides the three-dimensional structure of the truncated recombinant M-CSF a dimer as identified by the alpha-carbon positions of the amino acid residues in the sequence.

Specific residues in helices A, C, and D appear to be involved in the specificity of the receptor-binding interaction. Since M-CSFβ has intrachain disulfide bonds involving cysteines 157 and/or 159, the C-terminal region of M-CSF likely extends from the “rear” of the structure, providing a variable-length “tether” for membrane-bound forms of M-CSF. Thus, the “front” or receptor-binding region of M-CSF is on the opposite side of the molecules, consisting of solvent-accessible residues in or near helices A, C, and D, including residues from about 6 to 26, 71 to 90, and 110 to 130, respectively, of native M-CSF. Altering solvent accessible residues in these regions by site directed mutagenesis to increase or decrease side-chain interactions with the receptor may generate M-CSF agonists or antagonists. Residues having a solvent accessible surface area of greater than about 0.25 and preferably greater than about 0.4 are preferred based on normalization of the surface area of the amino acid accessible when in the trypeptide gly-x-gly (Kabsch, W. et al., Biopolymers 22:2577 (1983)) Preferably residues are chosen which do not interact with other parts of the protein such as the dimer interface in order to maintain the relative orientation of monomers and to avoid disturbing the process of protein folding. An optional additional consideration is selecting residues not conserved between human and mouse M-CSF, which does not recognize the human M-CSF receptor. Candidate amino acids are preferably selected for substitution with non-conservative amino acids, so as to disrupt hydrogen bonding and/or hydrophobic interactions with MCSF-R residues. For example, changing one or more histidines to non-hydrogen-donor amino acids of similar size may create an M-CSF with altered receptor binding ability. Preferred amino acids for substitution include but are not limited to: H15; Q79; R86; E115; E41; K93; D99; L55; S18; Q20; I75; V78; L85; D69; N70; H9; N63; and T34. M-CSF residues important in receptor signaling are believed to be composed of discontinuous regions of M-CSF. To minimize the likelihood of antibody formation to potentially administered M-CSF-based proteinaceous drugs, it is desirable to retain the solvent-accessible parental M-CSF residues (to resemble the native molecule) whenever possible.

Mutagenesis of amino acids H15 and H9 in the N-terminal/A helix region resulted in muteins with significantly lower biological activity and significantly lower MCSF-R binding ability. These results indicated that the reduced biological activity was due to decreased receptor binding affinity; thus, these histidine amino acids represent contacts that are important for M-CSF receptor binding affinity and should be left unchanged if full receptor-binding ability is desired. Nearby solvent accessible residues such as Y6 and S13 and others may also represent M-CSF receptor contact residues. A double mutant of M-CSF (Q20A, V78K) was constructed to test the importance of solvent accessible residues in the central portion of helices A and C. This double mutein had slightly lower (8-10 fold) biological activity and correspondingly lower receptor-binding activity. Mutagenesis of residues Q17, R21, E115 and E119 changed side chain properties of solvent-accessible amino acids in the areas of interest but did not affect biological specific activity, suggesting that these residues need not be altered in muteins designed to have antagonist activity.

In one embodiment, the invention contemplates use of M-CSF muteins in which residues of helices A and/or C and/or D involved in receptor-binding (for example, amino acids 6 to 26, 71 to 90 and/or 110 to 130) have been mutated non-conservatively. Such muteins preferably retain at least 65%, 70%, 75%, 80%, 85% or 90% similarity (i.e. amino acids that are identical or have similar properties) to the native sequence within helices A, C or D, but have higher similarity to the native sequence in the remainder of the polypeptide, e.g., at least 95%, 98% or 99% similarity. In addition, residues that support the three-dimensional confirmation of the receptor-binding site may be mutated non-conservatively.

In another embodiment, the M-CSF mutein is a monomeric form of M-CSF. The dimeric form of M-CSF is the biologically active form, and monomeric forms of M-CSF are generally not active. Disulfide bonding of the monomers appears to occur through the Cys31-Cys31 interchain linkage. Thus, it is contemplated that monomeric forms of M-CSF may be suitable for use as antagonists. Such forms include muteins comprising cysteine deletions and/or cysteine replacements (e.g., cysteine to alanine substitutions) of Cys31 and/or other cysteines, or muteins in which the cysteine(s), particularly Cys31, have been chemically modified so that they are not available for disulfide bonding.

In yet another embodiment, the M-CSF mutein comprises one or more of helices A, C or D, or portions thereof involved in receptor-binding, alone or fused to other polypeptides that allow display of the fragments in proper three-dimensional conformation.

Muteins containing any desired conservative and/or non-conservative muteins are readily prepared using techniques well known in the art, including recombinant production or chemical synthesis.

Conservative substitutions, particularly substitutions outside of regions directly involved in ligand-receptor binding, are not expected to significantly change the binding properties of the M-CSF muteins (or M-CSFR muteins). Amino acids can be classified according to physical properties and contribution to secondary and tertiary protein structure. A conservative substitution is recognized in the art as a substitution of one amino acid for another amino acid that has similar properties. Exemplary conservative substitutions are set out in Table 2 (from WO 97/09433, page 10, published Mar. 13, 1997 (PCT/GB96/02197, filed Sep. 6, 1996), immediately below.

TABLE 2
Conservative Substitutions I

| | |
|---|---|
| SIDE CHAIN | |
| CHARACTERISTIC | AMTNO ACID |

| | |
|---|---|
| Aliphatic | |
| Non-polar | G A P I L V |
| Polar-uncharged | C S T M N Q |
| Polar-charged | D E K R |
| Aromatic | H F W Y |
| Other | N Q D E |

Alternatively, conservative amino acids can be grouped as described in Lehninger, (Biochemistry, Second Edition; Worth Publishers, Inc. NY:NY (1975), pp. 71-77) as set out in Table 3, immediately below.

TABLE 3
Conservative Substitutions II

| SIDE CHAIN | |
|---|---|
| CHARACTERISTIC | AMINO ACID |
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

As still an another alternative, exemplary conservative substitutions are set out in Table 4, immediately below.

TABLE 4
Conservative Substitutions III

| | |
|---|---|
| Original Residue | Exemplary Substitution |

| | |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

The availability of a DNA sequence encoding M-CSF permits the use of various expression systems to produce the desired polypeptides. Construction of expression vectors and recombinant production from the appropriate DNA sequences are performed by methods well known in the art. These techniques and various other techniques are generally performed according to Sambrook et al., Molecular Cloning—A Laboratory Manual, Cold Spring Harbor Laboratory, Cold Spring Harbor, N.Y. (1989), and Kriegler, M., Gene Transfer and Expression, A Laboratory Manual, Stockton Press, New York (1990), both of which are incorporated herein by reference.

Certain modifications to the primary sequence of M-CSF can be made by deletion, addition, or alteration of the amino acids encoded by the DNA sequence without destroying the desired structure (e.g., the receptor binding ability of M-CSF) in accordance with well-known recombinant DNA techniques. Further, a skilled artisan will appreciate that individual amino acids may be substituted or modified by oxidation, reduction or other modification, and the polypeptide may be cleaved to obtain fragments that retain the active binding site and structural information. Such substitutions and alterations result in polypeptides having an amino acid sequence which falls within the definition of polypeptide “having substantially the same amino acid sequence as the mature M-CSFα (SEQ ID NO: 7), M-CSFβ (SEQ ID NO: 8), and M-CSFγ (SEQ ID NO: 9)polypeptides.”

Polypeptides may be produced by chemical synthesis or recombinant production techniques known in the art.

The relatedness of proteins can also be characterized through the relatedness of their encoding nucleic acids. Methods to determine identity and/or similarity of polynucleotide sequences are described above. In addition, methods to determine similarity of polynucleotide sequences through testing their ability to hybridize under moderately or highly stringent conditions may be determined as follows. Exemplary moderately stringent hybridization conditions are as follows: hybridization at 42° C. in a hybridization solution comprising 50% formamide, 1% SDS, 1 M NaCl, 10% Dextran sulfate, and washing twice for 30 minutes at 60° C. in a wash solution comprising 0.1×SSC and 1% SDS. Highly stringent conditions include washes at 68° C. in a wash solution comprising 0.1×SSC and 1% SDS. It is understood in the art that conditions of equivalent stringency can be achieved through variation of temperature and buffer, or salt concentration as described in the art (Ausubel, et al. (Eds.), Protocols in Molecular Biology, John Wiley & Sons (1994), pp. 6.0.3 to 6.4.10). Modifications in hybridization conditions can be empirically determined or precisely calculated based on the length and the percentage of guanosine/cytosine (GC) base pairing of the probe. The hybridization conditions can be calculated as described in Sambrook et al., (Eds.), Molecular Cloning: A Laboratory Manual, Cold Spring Harbor Laboratory Press: Cold Spring Harbor, N.Y. (1989), pp. 9.47 to 9.51.

C. Soluble M-CSFR

Exemplary M-CSFR fragments according to the invention may comprise one or more, or two or more, of domains involved in M-CSF/receptor binding (believed to be domains 1, 2 and 3). Preferred M-CSFR fragments comprises all three of domains 1, 2 and 3 of M-CSFR. Additional mutations and/or modifications to such fragments or to the entire extracellular domain of M-CSFR are contemplated and may be produced as described above in the section on M-CSF muteins.

M-CSFR (SEQ ID NOs: 84 and 85) is a membrane spanxing molecule with five extracellular immunoglobulin-like domains (of which domains 1-3 are believed to be involved in ligand-receptor binding), a transmembrane domain and an intracellular interrupted Src related tyrosine kinase domain. With reference to SEQ ID NO: 85, the aforementioned domains are located as follows: Ig domain 1: amino acids 27-102; Ig domain 2: amino acids 112-196; 1 g domain 3: amino acids 215-285; Ig domain 4: amino acids 308-399; Ig domain 5: amino acids 410-492; transmembrane domain: amino acids 515-537; and kinase domain: amino acids 582-910. A “typical” immunoglobulin-like domain contains a loop structure usually anchored by a disulfide bond between two cysteines at the extremity of each loop. In M-CSF-R, these cysteines forming the Ig-like loops are at the following amino acid positions: Domain 1: 42, 84; Domain 2: 127, 177; Domain 3: 224, 278; Domain 4: no cysteins involved; Domain 5: 419, 485.

The intact extracellular portion of M-CSFR or any fragment thereof that retains antigenicity, for example, one or more of the Ig-like loops, may be used to raise antibodies that would bind to the native receptor. Polyclonal, monoclonal, chimeric, CDR grafted, humanized, fully human antibodies and antigen-binding fragments thereof may be prepared as described above for antibodies to M-CSF. The antibody products may be screened for activity as an MCSF antagonist and for suitability in the treatment methods of the invention using assays as described in the section entitled “Screening Methods” herein or using any suitable assays known in the art.

One or more of the aforementioned Ig-like loops within the extracellular domain of the receptor may be sufficient to inhibit interaction between M-CSF and M-CSFR. Thus fragments of the extracellular domain of M-CSFR and muteins thereof may be easily prepared using recombinant or chemical synthetic means well known in the art. The products may be screened for activity as an MCSF antagonist and for suitability in the treatment methods of the invention using assays as described in the section entitled “Screening Methods” herein or using any suitable assays known in the art.

D. Gene Therapy

Delivery of a therapeutic protein to appropriate cells can be effected via gene therapy ex vivo, in situ, or in vivo by use of any suitable approach known in the art, including by use of physical DNA transfer methods (e.g., liposomes or chemical treatments) or by use of viral vectors (e.g., adenovirus, adeno-associated virus, or a retrovirus). Antiserse compounds and methods of using them are also provided by the present invention. The level of M-CSF or M-CSFR activity may be reduced by using well-known antisense, gene “knock-out,” ribozyme, triple helix or RNAi methods to decrease the level gene expression. Techniques for the production and use of such molecules are well known to those of skill in the art.

As used herein, the term “peptidomimetic” is a non-peptide compound that comprises an assembly of amino acid side chains, or pharmacophores, or suitable derivatives thereof, that are supported on a scaffold such that the spatial orientation of the pharmacophores substantially mimic the bioactive conformation of a natural peptide. For example, a peptidomimetic may lack amino acids or peptide bonds but retain the particular three-dimensional arrangement of peptide chain groups from the parent peptide that is required for binding activity. The scaffold may comprise a bicyclic, tricyclic or higher polycyclic carbon or heteroatom skeleton, or may be based on one or more ring structures (e.g., pyridine, indazole, etc.) or amide bonds. This scaffold may be linked by spacers to an acidic group (e.g. a carboxylic acid functional group) at one end and a basic group (e.g. an N-containing moiety such as amidine or guanidine) at the other end of the core. Exemplary techniques for synthesizing peptidomimetics are described in U.S. patent application no. 20030199531 published Oct. 23, 2003, U.S. Patent Application No. 20030139348 published Jul. 24, 2003.

In addition to antibodies and other proteins, this invention also contemplates alternative M-CSF antagonists including, but not limited to, peptides or small organic molecules that are also effective in inhibiting the interaction between M-CSF and M-CSFR or the activation of M-CSFR.

II. Combination Therapy

Concurrent administration of two therapeutic agents according to the present invention, such as an M-CSF antagonist and a second anti-osteoclast agent, does not require that the agents be administered at the same time or by the same route, as long as there is an overlap in the time period during which the agents are exerting their therapeutic effect. Simultaneous or sequential administration is contemplated, as is administration on different days or weeks.

The discovery of a significant time lag to observe therapeutic effect after commencing treatment with an M-CSF antibody (an exemplary M-CSF antagonist) makes desirable the co-administration of a second anti-osteoclast agent with quicker onset of action during this transition period. During the transition period, the two agents must be administered at a monotherapeutically effective amount. Subsequent to the transition period, the second anti-osteoclast agent may be discontinued or reduced in dosage. If the M-CSF antagonist and second anti-osteoclast agent exert synergistic effects, the dose of one or both may be lowered after the transition period.

Compositions of the invention are administered to a mammal already suffering from, or predisposed to, osteolytic disorder, including cancer metastasis and/or bone loss associated with cancer metastasis, or other bone loss related diseases, such as osteoporosis, in an amount sufficient to prevent or at least partially arrest the development of such disease. An amount of a therapeutic agent adequate to accomplish this when the therapeutic agent is given alone (not in combination with a second therapeutic agent) is defined as a “monotherapeutically effective dose.”

In the combination therapy methods of the present invention, the M-CSF antagonist, such as the M-CSF antibody, and the second anti-osteoclast agent may be administered simultaneously or at different time. The two agents can be administered, for example, within 8 hours, 1 day, 14 days, 30 days, 3 months, 6 months, 9 months or 1 year of each other.

Exemplary second anti-osteoclast agents include bisphosphonates, including but not limited to zoledronate, pamidronate, clodronate, etidronate, tiludronate, alendronate, ibandronate or risedronate. Exemplary other anti-osteoclast agents include bisphosphonates, PTHrP neutralizing agents (e.g., antibody, antisense, siRNA), cathepsin K inhibitors, MIP-1-α antagonists, RANK/RANKL neutralizing agents (e.g., anti-RANK antibody, such as AMG-162, or antisense, soluble RANKL receptor or muteins thereof), RANKL vaccine, osteoprotegrin (OPG), platelet-derived growth factors (PDGF), src kinase inhibitors, gallium maltolate, and matrix metalloproteinase (MMP) inhibitors.

Exemplary doses of bisphosphonates include the intravenous administration of 4 mg. Lesser dosages may also be administered including 3.5 mg, 3.3 mg or 3.0 mg. Other routes of administration are possible including subcutaneous and as described in WO 02/087555. Effective amounts of a M-CSF antibody will vary and depend on the severity of the disease and the weight and general state of the patient being treated, but generally range from about 1.0 mg/kg to about 100 mg/kg body weight, or about 10 mg/kg to about 30 mg/kg, with dosages of from about 0.1 mg/kg to about 10 mg/kg or about 1 mg/kg to about 10 mg/kg per application being more commonly used. For example, about 10 11 g/kg to 5 mg/kg or about 30 Mg/kg to 1 mg/kg of antibody is an initial candidate dosage for administration to the patient, whether, for example, by one or more separate administrations, or by continuous infusion. Administration is daily, on alternating days, weekly or less frequently, as necessary depending on the response to the disease and the patient's tolerance of the therapy. Maintenance dosages over a longer period of time, such as 4, 5, 6, 7, 8, 10 or 12 weeks or longer may be needed until a desired suppression of disease symptoms occurs, and dosages may be adjusted as necessary. The progress of this therapy is easily monitored by conventional techniques and assays.

Although the methods of the present invention may be useful for all stages of cancers, they may be particularly appropriate in advanced or metastatic cancers. Combining the therapy method with a chemotherapeutic or radiation regimen may be preferred in patients that have not received chemotherapeutic treatment, whereas treatment with the therapy method of the present invention may be indicated for patients who have received one or more chemotherapies. Additionally, the therapy methods of the present invention can also enable the use of reduced dosages of concomitant chemotherapy, particularly in patients that do not tolerate the toxicity of the chemotherapeutic agent very well.

The method of the invention contemplate the administration of single anti-M-CSF antibodies, as well as combinations, or “cocktails”, of different antibodies. Such antibody cocktails may have certain advantages in as much as they contain antibodies which exploit different effector mechanisms or combine directly cytotoxic antibodies with antibodies that rely on immune effector functionality. Such antibodies in combination may exhibit synergistic therapeutic effects.

The methods of the invention can be used in combination with yet other therapeutics, such as cancer therapeutics. Exemplary cancer therapeutic agents and/or procedures, include but are not limited to various chemotherapeutic agents, androgen-blockers, and immune modulators (e.g., IL-2, GM-CSF, SLC), Bisphosphonate(s) (e.g., Aredia (i.e., pamidronate, pamidronic acid, disodium pamidronate, pamidronate disodium pentahydrate); Zometa (i.e., Aclasta, zoledronic acid, zoledronate); Clondronate (i.e., Bonefos, Loron, clodronate disodium, sodium clondronate); Fosamax (i.e., alendronate, alendronate sodium salt trihydrate, alendronic acid); Fosavance (i.e., Fosamax formulated with vitamin D); Bondronat or Bonviva or Boniva (i.e., ibandronate, ibandronic acid, ibandronate sodium); Actonel (i.e., risedronate, risedronate sodium, risendronic acid); Didronel or Didrocal (i.e., etidronate, etidronic acid, etidronate disodium); Nerixia (i.e., neridronate, neridronic acid); Skelid (i.e., tiludronate, tiludronic acid); dimethyl-APD (i.e., olpadronate, olpadronic acid); and medronic acid or medronate), surgery, radiation, cytotoxic chemotherapy, hormone therapy (e.g., Tamoxifen; anti-Androgen therapy), antibody therapy (e.g., antibodies to RANKL/RANK neutralizing; PTHrP neutralizing, anti-Her2, anti-CD20, anti-CD40, CD22, VEGF, IGFR-1, EphA2, HAAH, TMEFF2, CAIX antibodies), therapeutic protein therapy (e.g., soluble RANKL receptor; OPG, and PDGF and MMP inhibitors), small molecule drug therapy (e.g., Src-kinase inhibitor), kinase inhibitors of growth factor receptors, or RANKL inhibitors, oligonucleotides therapy (e.g., RANKL or RANK or PTHrP Anti-sense), gene therapy (e.g. RANKL or RANK inhibitors, such as anti-RANKL antibodies), peptide therapy (e.g. muteins of RANKL) as well as those proteins, peptides, compounds, and small molecules described herein.

Cancer chemotherapeutic agents include, without limitation, alkylating agents, such as carboplatin and cisplatin; nitrogen mustard alkylating agents; nitrosourea alkylating agents, such as carmustine (BCNU); antimetabolites, such as methotrexate; folinic acid; purine analog antimetabolites, mercaptopurine; pyrimidine analog antimetabolites, such as fluorouracil (5-FU) and gemcitabine (Gemzar®); hormonal antineoplastics, such as goserelin, leuprolide, and tamoxifen; natural antineoplastics, such as aldesleukin, interleukin-2, docetaxel, eloposide (VP-16), interferon alfa, paclitaxel (Taxol®), and tretinoin (ATRA); antibiotic natural antineoplastics, such as bleomycin, dactinomycin, daunorubicin, doxorubicin, daunomycin and mitomycins including mitomycin C; and vinca alkaloid natural antineoplastics, such as vinblastine, vincristine, vindesine; hydroxyurea; aceglatone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide (Cytoxin®); Schizophyllan, cytarabine (cytosine arabinoside), dacarbazine, thioinosine, thiotepa, tegafur, dolastatins, dolastatin analogs such as auristatin, CPT-11 (irinotecan), mitozantrone, vinorelbine, teniposide, aminopterin, caminomycin, esperamicins (See, e.g., U.S. Pat. No. 4,675,187), neocarzinostatin, OK-432, bleomycin, furtulon, broxuridine, busulfan, honvan, peplomycin, bestatin (Ubenimex®), interferon-β, mepitiostane, mitobronitol, melphalan, laminin peptides, lentinan, Coriolus versicolor extract, tegafur/uracil, estramustine (estrogen/mechlorethamine).

Further, additional agents used as therapy for cancer patients include EPO, G-CSF, ganciclovir; antibiotics, leuprolide; meperidine; zidovudine (AZT); interleukins 1 through 18, including mutants and analogues; interferons or cytokines, such as interferons α, β, and γ hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; anti-angiogenesis factors: antigenic materials; and pro-drugs.

Prodrug refers to a precursor or derivative form of a pharmaceutically active substance that is less cytotoxic or non-cytotoxic to tumor cells compared to the parent drug and is capable of being enzymatically activated or converted into an active or the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). Prodrugs include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use herein include, but are not limited to, those chemotherapeutic agents described above.

III. Administration and Preparation

Effective amounts of a M-CSF antagonist will vary and depend on the severity of the disease and the weight and general state of the patient being treated, but generally range from about 1.0 μg/kg to about 100 mg/kg body weight, with dosages of from about 10 μg/kg to about 10 mg/kg per application being more commonly used. Determination of an effective amount of a composition of the invention can be accomplished through standard empirical methods which are well known in the art. For example, the in vivo neutralizing activity of sera from a subject treated with a given dosage of M-CSF antagonist may be evaluated using an assay that determines the ability of the sera to block M-CSF induced proliferation and survival of murine monocytes (CD11b+ cell, a subset of CD11 cells, which expresses high levels of receptor to M-CSF) in vitro as described in Cenci et al., J. Clin. Invest. 1055: 1279-87, 2000.

Administration is daily, every two days, every 3 days, twice weekly, weekly or less frequently, as necessary depending on the response to the disease and the patient's tolerance of the therapy. Maintenance dosages over a prolonged period of time may be needed, and dosages may be adjusted as necessary.

Single or multiple administrations of the compositions can be carried out with the dose levels and pattern being selected by the treating physician.

The M-CSF antagonists, including anti-M-CSF antibodies used in the practice of a method of the invention may be formulated into pharmaceutical compositions comprising a carrier suitable for the desired delivery method. Suitable carriers include any material which, when combined with the M-CSF antagonist, retains the anti-tumor function of the antagonist and is nonreactive with the subject's immune systems. Examples include, but are not limited to, any of a number of standard pharmaceutical carriers such as sterile phosphate buffered saline solutions, bacteriostatic water, and the like. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

Therapeutic formulations of the antagonist are prepared for storage by mixing the antagonist having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium-chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpylrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEENT, PLURONICS™ or polyethylene glycol (PEG).

The formulation herein may also contain more than one active compound as necessary for the particular indication being treated, preferably those with complementary activities that do not adversely affect each other. For example, it may be desirable to further provide an immunosuppressive agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

The antagonist is administered by any suitable means, including parenteral, subcutaneous, intraperitoneal, intrapulmonary, and intranasal, and, if desired for local treatment, intralesional administration. Parenteral infusions include intravenous, intraarterial, intraperitoneal, intramuscular, intradermal or subcutaneous administration. In addition, the antagonist is suitably administered by pulse infusion, particularly with declining doses of the antagonist. Preferably the dosing is given by injections, most preferably intravenous or subcutaneous injections, depending in part on whether the administration is brief or chronic. Other administration methods are contemplated, including topical, particularly transdermal, transmucosal, rectal, oral or local administration e.g. through a catheter placed close to the desired site.

Compositions of the present invention can be in the form of, for example, granules, powders, tablets, capsules, syrup, suppositories, injections, emulsions, elixirs, suspensions or solutions. The instant compositions can be formulated for various routes of administration, for example, by oral administration, by nasal administration, by rectal administration, subcutaneous injection, intravenous injection, intramuscular injections, or intraperitoneal injection.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Sustained-release preparations may be prepared. Suitable examples of sustained-release preparations include semipermeable matrices of solid hydrophobic polymers containing the antagonist, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antagonists remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The formulations of the invention may be designed to be short-acting, fast-releasing, long-acting, or sustained-releasing as described herein. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Besides those representative dosage forms described above, pharmaceutically acceptable excipients and carries are generally known to those skilled in the all and are thus included in the instant invention. Such excipients and carriers are described, for example, in “Remingtons Pharmaceutical Sciences” Mack Pub. Co., New Jersey (1991), which is incorporated herein by reference.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

M-CSF antagonists or antibodies useful as therapeutics according to the invention will often be prepared substantially free of other naturally occurring immunoglobulins or other biological molecules. Preferred M-CSF antagonists will also exhibit minimal toxicity when administered to a mammal afflicted with, or predisposed to suffer from, osteolytic disorders, including cancer metastasis and/or bone loss associated with cancer metastasis.

The compositions of the invention may be sterilized by conventional, well known sterilization techniques. The resulting solutions may be packaged for use or filtered under aseptic conditions and lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride and stabilizers (e.g., 120% maltose, etc.).

The M-CSF antagonists of the present invention may also be administered via liposomes, which are small vesicles composed of various types of lipids and/or phospholipids and/or surfactant which are useful for delivery of a drug (such as the antagonists disclosed herein and, optionally, a chemotherapeutic agent). Liposomes include emulsions, foams, micelles, insoluble monolayers, phospholipid dispersions, lamellar layers and the like, and can serve as vehicles to target the M-CSF antagonists to a particular tissue as well as to increase the half life of the composition. A variety of methods are available for preparing liposomes, as described in, e.g., U.S. Pat. Nos. 4,837,028 and 5,019,369, which patents are incorporated herein by reference.

Liposomes containing the antagonist are prepared by methods known in the art, such as described in Epstein et al., Proc. Natl. Acad. Sci. USA 82: 3688 (1985); Hwang et al., Proc. Natl Acad. Sci. USA 77: 4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the M-CSF antibody of the present invention can be conjugated to the liposomes as described in Martin et al., J. Biol. Chem. 257: 286-288 (1982) via a disulfide interchange reaction. A chemotherapeutic agent (such as Doxorubicin) is optionally contained within the liposome [see, e.g., Gabizon et al., J. National Cancer Inst. 81(19): 1484 (1989)].

The concentration of the M-CSF antagonist in these compositions can vary widely, i.e., from less than about 10%, usually at least about 25% to as much as 75% or 90% by weight and will be selected primarily by fluid volumes, viscosities, etc., in accordance with the particular mode of administration selected. Actual methods for preparing orally, topically and parenterally administrable compositions will be known or apparent to those skilled in the art and are described in detail in, for example, Remington's Pharmaceutical Science, 19th ed., Mack Publishing Co., Easton, Pa. (1995), which is incorporated herein by reference.

Determination of an effective amount of a composition of the invention can be accomplished through standard empirical methods which are well known in the art. For example, the in vivo neutralizing activity of sera from a subject treated with a given dosage of M-CSF antagonist may be evaluated using an assay that determines the ability of the sera to block M-CSF induced proliferation and survival of murine monocytes (CD11b+cell, a subset of CD11 cells, which expresses high levels of receptor to M-CSF) in vitro as described in Cenci et al., J. Clin. Invest. 1055: 1279-87, 2000.

Compositions of the invention are administered to a mammal already-suffering from, or predisposed to, osteolytic disorder, including cancer metastasis and/or bone loss associated with cancer metastasis in an amount sufficient to prevent or at least partially arrest the development of such disease. Effective amounts of a M-CSF antagonist will vary and depend on the severity of the disease and the weight and general state of the patient being treated, but generally range from about 1.0 mg/kg to about 100 mg/kg body weight, or about 10 mg/kg to about 90 mg/kg, with dosages of from about 20 mg/kg to about 80 mg/kg or about 30 mg/kg to about 70 mg/kg or about 40 mg/kg to about 60 mg/kg per application. For example, about 10 mg/kg to 50 mg/kg or about 20 mg/kg to 60 mg/kcg of anti-MCSF antibody is an initial candidate dosage for administration to the patient, whether, for example, by one or more separate administrations, or by continuous infusion. Administration is daily, on alternating days, weekly or less frequently, as necessary depending on the response to the disease and the patient's tolerance of the therapy. Maintenance dosages over a longer period of time, such as 4, 5, 6, 7, 8, 10 or 12 weeks or longer may be needed until a desired suppression of disease symptoms occurs, and dosages may be adjusted as necessary. The progress of this therapy is easily monitored by conventional techniques and assays.

Single or multiple administrations of the compositions can be carried out with the dose levels and pattern being selected by the treating physician. For the prevention or treatment of disease, the appropriate dosage of M-CSF antagonist, including anti-M-CSF antibody will depend on the type of disease to be treated, as defined above, the severity and course of the disease, whether the antagonist is administered for preventive or therapeutic purposes, previous therapy, the patient's clinical history and response to the antagonist, and the discretion of the attending physician. The antagonist is suitably administered to the patient at one time or over a series of treatments.

The antagonist composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutically effective amount of the antagonist to be administered will be governed by such considerations, and is the minimum amount necessary to prevent, ameliorate, or treat the M-CSF mediated disease, conditioner disorder, particularly to treat cancer cells, and most particularly to treat tumor cell metastases. Such amount is preferably below the amount that is toxic to the host or renders the host significantly more susceptible to infections.

In another embodiment of the invention, an article of manufacture containing materials useful for the treatment of the diseases, disorders or conditions described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the M-CSF antagonist or antibody of the invention. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further other materials desirable from a commercial and user standpoint, including other buffers, diluents, filters, needles, syringes, and package inserts with instructions for use.

The invention is illustrated by the following examples, which are not intended to be limiting in any way.

### EXAMPLES

### Example 1

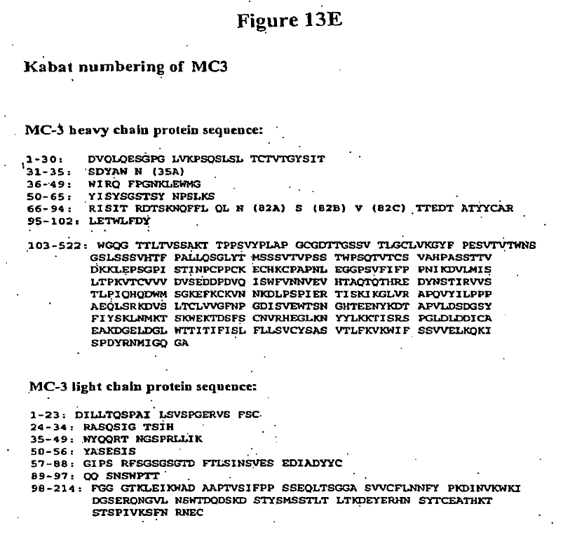

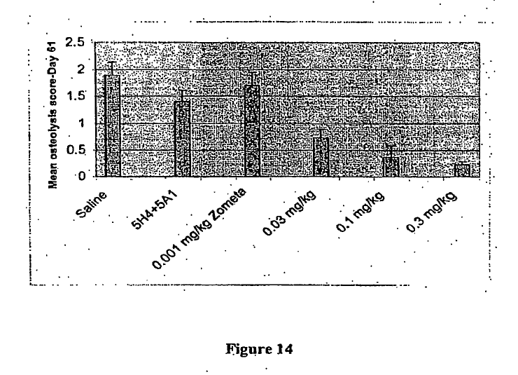

This Example establishes the dose-dependent, anti-resorptive effects of Zometa (zoledronate) in an animal model (FIG. 14). Treatment with ≧0.03 mg/kg Zometa inhibited the osteolytic damage caused by tumor growth at the bony site. In addition, a dose response effect was observed when mice were treated with increasing concentrations of Zometa. Anti-mouse and anti-human M-CSF mAbs 5A1 and 5H4 combined also protected against bone damage. M-CSF antibody alone was more effective than 0.03 mg/kg Zometa in treating severe osteolytic damage. Osteolysis score based on x-ray image in last-day of study (FIG. 14):

0=normal;

1=Equivocal or minimal lesion with normal cortex architecture;

2=Definite lytic lesion with minimal cortex/architecture disruption

3=Large lesion(s) with cortex/Architecture disruption

4=Gross destruction with no preserved architecture

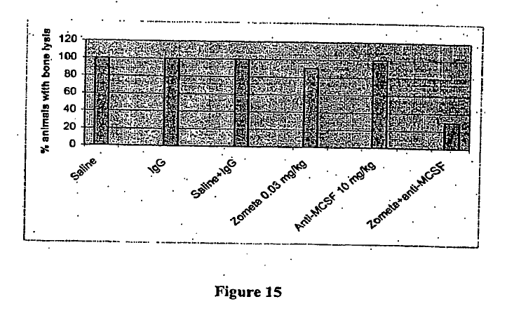

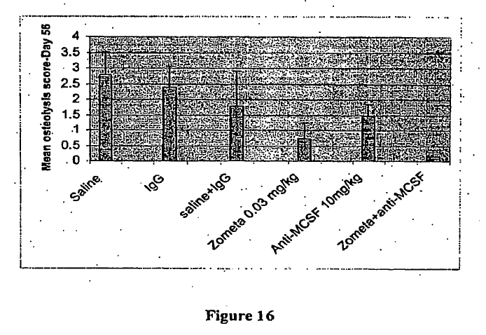

From this initial study, a combination study was performed using a sub-efficacious dose of 0.03 mg/kg Zometa. Two weeks after intratibial inoculation of 6×10⁵ MDA-MB-231Luc cells, Nu/Nu mice were treated with 5A1/5B4 (10 mg/kg once weekly), Zometa (0.03 mg/kg twice weekly) or both antibody and bisphosphonate. Bone lesions were monitored weekly by Faxitron analysis (x-ray technology), and at the end of the study, all animals were subjected to a final x-ray, and the images collected and distributed for scoring of the severity of lesions. Analysis of the results showed that both anti-MCSF mAb and Zometa were effective in treating osteolysis, but that the combined treatment of Zometa and the M-CSF mAb inhibited incidence (FIG. 15) and extent (FIG. 16) of bone lysis to a greater extent than either treatment alone.

FIG. 16 shows that treatment with 0.03 mg/kg Zometa or 10 mg/kg anti-MCSF antibody (5A1+5H4) inhibited the osteolytic damage caused by tumor growth at the bony site. A combination regimen of Zometa plus anti-MCSF antibody further inhibited bone lysis. Mean osteolysis scores were calculated from 1) an average of scores from 3 separate volunteer scorers and 2) a group average (originally 10 animals/group).

Osteolysis score based on x-ray image in last day of study:

0=normal;

1=Equivocal or minimal lesion with normal cortex architecture;

2=Definite lytic lesion with minimal cortex/architecture disruption

3=Large lesion(s) with cortex/Architecture disruption

4=Gross destruction with no preserved architecture

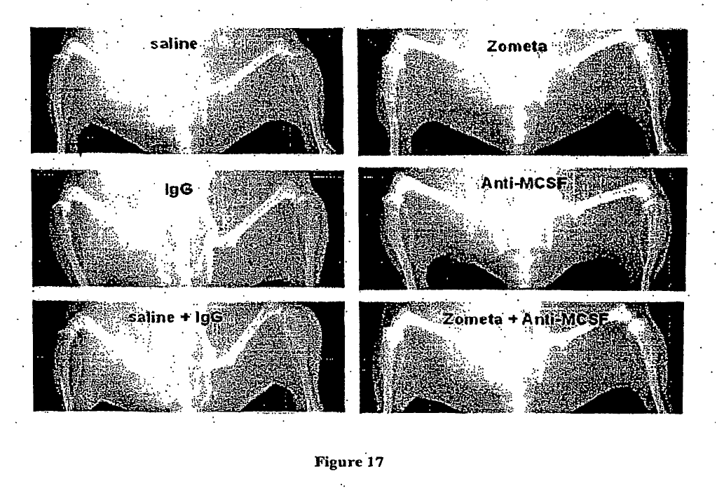

Examination of representative faxitron images further demonstrated the severity of lesions found in untreated animals compared to the relatively minor lesions in Zometa and anti-MCSF antibody-treated animals (FIG. 17). There were no adverse interactions observed in the combination treatment group.

In conclusion, both anti-MCSF antibody and Zometa effectively inhibit osteolysis, and combining the two treatments results in an increased anti-resorptive effect compared with either treatment alone. This suggests that the combination may be a safe and effective option for patients with bone disease who are either bisphosphonate-intolerant, or who are already being treated with bisphosphonates.

### Example 2

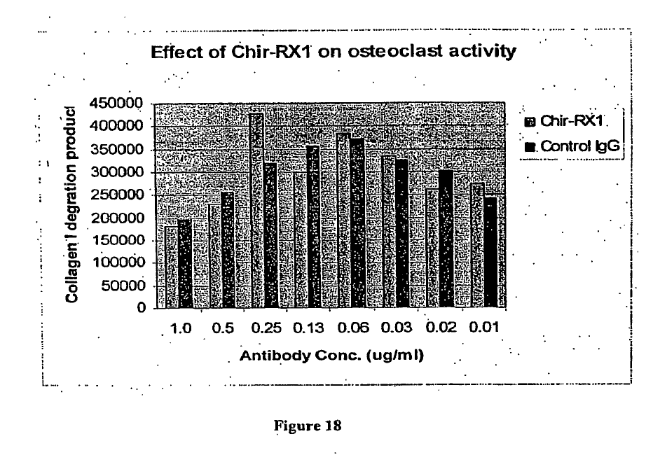

This Example shows that inhibition of M-CSf activity has no effect on differentiated osteoclasts activity (FIG. 18). The effect of M-CSF-neutralizing antibodies and bisphosphonate on differentiated osteoclast activity was tested with humanized Chir-RX1 and Zometa.

The human bone marrow CD34+ cells (Biowhittaker catalog number 2M-101 A, 3×10⁵ cells/vial) were induced to differentiate into osteoclasts under the experimental conditions described here. On Day 1, CD34+ cells were thawed from one frozen vial into 10 ml of media (Alpha MEM with 10% FCS, 1×Pen Strep and 1×fungizone) The cells were washed once and re-suspended in 2 ml of media and plate into onto the OsteoLyse plate (OsteoLyse™ Assay Kit (Human Collagen), Cambrex) at 100 ul per well. On day 2, without removing the original media, add to each well 50 ul of 4×CSF-1 to 30 ng/ml final concentration and 50 ul of 4×RANKL (sRANKL, Chemicon catalog #GF091, 10 ug/package) to final concentration of 100 ng/ml. On day 7, add to each well 50 ul of 5×RANKL to final concentration of 100 ng/ml.

On day 15, antibodies (either Chir-RX1 or control antibody) or Zometa were added at the indicated concentrations. On day 17, 10 ul of supernatant of the cell culture was sampled and mixed with 200 μl of Fluorophore Releasing Reagent in each well of the black 96-well assay plate (included in the OsteoLyse Assay Kit).

### Example 3

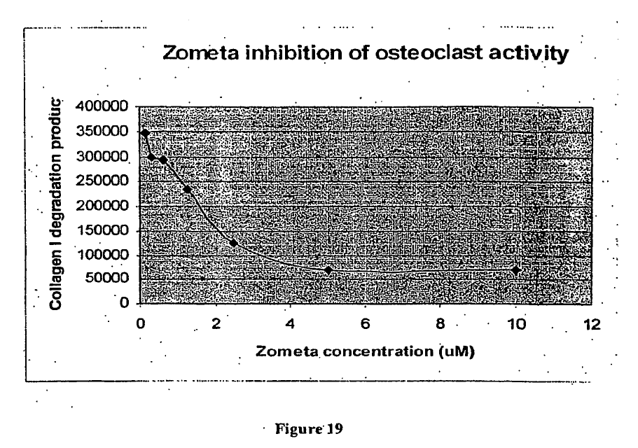

This Example shows that Zometa inhibits differentiated osteoclast activity in a dose-dependent manner (FIG. 19). The effect of M-CSF-neutralizing antibodies and bisphosphonate on differentiated osteoclast activity was tested with humanized Chir-RX1 and Zometa.

The human bone marrow CD34+ cells (Biowhittaker catalog number 2M-101 A, 3×10⁵ cells/vial) were induced to differentiate into osteoclasts under the experimental conditions described here. On Day 1, CD34+ cells were thawed from one frozen vial into 10 ml of media (Alpha MEM with 10% FCS, 1×Pen Strep and 1×fungizone). The cells were washed once and re-suspended in 2 ml of media and plate into onto the OsteoLyse plate (OsteoLyse™ Assay Kit (Human Collagen), Cambrex) at 100 ul per well. On day 2, without removing the original media, add to each well 50 ul of 4×CSF-1 to 30 ng/ml final concentration and 50 ul of 4×RANKL (sRANKL, Chemicon catalog #GF09, 10 ug/package) to final concentration of 100 ng/mil. On day 7, add to each well 50 ul of 5×RANKL to final concentration of 100 ng/ml.

On day 15, antibodies (either Chir-RX1 or control antibody) or Zometa vere added at the indicated concentrations. On day 17, 10 ul of supernatant of the cell culture was sampled and mixed with 200 μl of Fluorophore Releasing Reagent in each well of the black 96-well assay plate (included in the OsteoLyse Assay Kit).

### Example 4

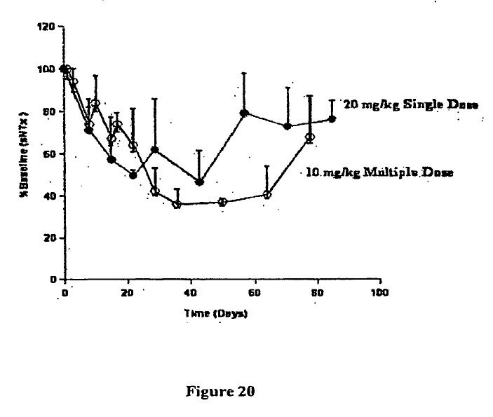

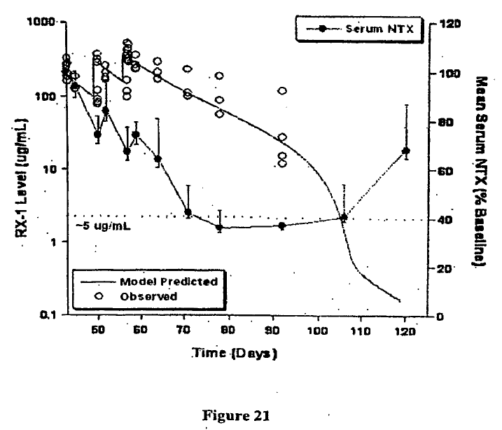

This Example shows the results of a pharmacokinetic and pharmacodynamic study using RX1 in primates (FIG. 20 and FIG. 21).

The purpose of this study was to investigate the pharmacodynamics and pharmacokinetics of heRX1-10.G1, a humanized anti-human M CSF antibody, when administered to cynomolgus monkeys either as a single slow bolus intravenous injection (Groups 2 and 3 on Day 1) followed by a 13-week observation period or as repeated doses (Group 1 on Days 1, 43, 50, and 57) followed by a 10-week observation period. Humanized anti-M-CSF IgG1 monoclonal antibody was administered via slow bolus intravenous (IV) injection via a brachial or saphenous vein.

The use of animal is required by worldwide regulatory agencies for safety assessment of new drugs. The antibody is not cross-reactive in rodent species but has been shown to be active in cynomolgus monkeys. Therefore, the cynomolgus monkey was selected since it is an accepted non-rodent species for use in intravenous injection studies with biologics.

Animals were randomized into the following groups:

| Group No. | Dose Level | Dose Volume | No. of Animals | |
|---|---|---|---|---|
| Identification | (mg/kg) | (mL/kg) | Males | Females |
| 1 heRX1 - 10.G1 | 0.2/10 ^{a} | 4 | 2 | 2 |
| 2 heRX1 - 10.G1 | 2 | 4 | 2 | 2 |
| 3 heRX1 - 10.G1 | 20 | 4 | 2 | 2 |

^{a} On Day 1, Group 1 will receive a 0.2 mg/kg/dose, and on Days 43, 50, and 57, the same animals will receive a 10 mg/kg/dose.

Groups 2 and 3 were administered the test article formulation (2 and 20 mg/kg/dose, respectively) on Day 1 by intravenous slow bolus injection over an approximate 10-minute period. Formulations were administered via a saphenous vein using a catheter and an abbocath. The dose volume was 4 mL/kg and the actual dose was based on the most recent practical body weight of each animal. Group 1 was administered a dose of 0.2 mg/kg on Day 1 and subsequent doses of 10 mg/kg/dose on Days 43, 50 and 57. The formulation was administered by intravenous slow bolus injection (over an approximate 10 minute period) via a saphenous vein using a catheter and an abbocath. The dose volume was 4 mL/kg and the actual dose was based on the most recent practical body weight of each animal.

Blood was collected from all animals for hematology and/or clinical biochemistry, as follows:

| | Hematology | | | Biochemistry | | |
|---|---|---|---|---|---|---|
| Occasion | Group 1 | Group 2 | Group 3 | Group 1 | Group 2 | Group 3 |
| Day −14 ^{a} | 0.5 mL | 0.5 mL | 0.5 mL | 1 mL | 1 mL | 1 mL |
| Day −7 | 0.5 mL | 0.5 mL | 0.5 mL | | | |
| Day 3 ^{a} | 0.5 mL | 0.5 mL | 0.5 mL | | | |
| Day 8 | 0.5 mL | 0.5 mL | 0.5 mL | | | |
| Day 15 | 0.5 mL | 0.5 mL | 0.5 mL | 1 mL | 1 mL | 1 mL |
| Day 22 | 0.5 mL | 0.5 mL | 0.5 mL | | | |
| Day 29 | 0.5 mL | 0.5 mL | 0.5 mL | 1 mL | 1 mL | 1 mL |
| Day 43 | 0.5 mL | 0.5 mL | 0.5 mL | 1 mL | 1 mL | 1 mL |
| Day 50 | 0.5 mL | 0.5 mL | 0.5 mL | 1 mL | | |
| Day 57 | 0.5 mL | 0.5 mL | 0.5 mL | | 1 mL | 1 mL |
| Day 64 | 0.5 mL | 0.5 mL | 0.5 mL | 1 mL | | |
| Day 71 | 0.5 mL | 0.5 mL | 0.5 mL | | 1 mL | 1 mL |
| Day 78 | 0.5 mL | 0.5 mL | 0.5 mL | 1 mL | | |
| Day 85 | | 0.5 mL | 0.5 mL | | 1 mL | 1 mL |
| Day 92 | 0.5 mL | | | 1 mL | | |
| Day 106 | 0.5 mL | | | 1 mL | | |
| Day 120 | 0.5 mL | | | 1 mL | | |

^{a} Only WBC count (total and differential) required to correlate with lymphocyte phenotyping

The following parameters were examined:

Hematology: blood cell morphology; erythrocyte indices (MCV, MCH, MCHC and RDW); hematocrit; hemoglobin; mean platelet volume; platelet count; red blood cell count; reticulocytes (absolute and percent); and white blood cell count (total, absolute and percent differential).

Clinical biochemistry: A/G ratio (calculated); alanine aminotransferase; albumin; alkaline phosphatase; aspartate aminotransferase; blood urea nitrogen; calcium; chloride; cholesterol; creatinine; globulin (calculated); glucose; inorganic phosphorus; potassium; sodium; total, direct and indirect bilirubin; total protein; triglycerides; and C-reactive protein.

Biochemical markers of bone turnover were analyzed as follows (approximately 2 mL of blood was collected from all animals for determination of bone biomarkers):

| | Markers of Bone Formation | | | Markers of Bone Resorption | | |
|---|---|---|---|---|---|---|
| | (BAP and Calcium ^{#)} | | | (NTx and CTx) | | |
| Occasion | Group 1 | Group 2 | Group 3 | Group 1 | Group 2 | Group 3 |
| Day −14 | 1 mL | 1 mL | 1 mL | 1 mL | 1 mL | 1 mL |
| Day −7 | 1 mL | 1 mL | 1 mL | 1 mL | 1 mL | 1 mL |
| Day 8 | | | | 1 mL | 1 mL | 1 mL |
| Day 15 | | | | 1 mL | 1 mL | 1 mL |
| Day 22 | | | | 1 mL | 1 mL | 1 mL |
| Day 29 | 1 mL | 1 mL | 1 mL | 1 mL | 1 mL | 1 mL |
| Day 43 | | | | 1 mL | 1 mL | 1 mL |
| Day 45 | | | | 1 mL | | |
| Day 50 | 1 mL | | | 1 mL | | |
| Day 52 | | | | 1 mL | | |
| Day 57 | | 1 mL | 1 mL | 1 mL | 1 mL | 1 mL |
| Day 59 | | | | 1 mL | | |
| Day 64 | 1 mL | | | 1 mL | | |
| Day 71 | | | | 1 mL | 1 mL | 1 mL |
| Day 78 | | | | 1 mL | | |
| Day 85 | | 1 mL | 1 mL | | 1 mL | 1 mL |
| Day 92 | 1 mL | | | 1 mL | | |
| Day 106 | 1 mL | | | 1 mL | | |
| Day 120 | 1 mL | | | 1 mL | | |

(NTX: N-terminal cross-linking telopepide of bone collagen)
(CTX: C-terminal cross-linking telopepide of bone collagen)

Pharmacokinetic Evaluation and Serum M-CSF Activity

For Groups 2 and 3, blood (1.5 mL each) was collected by venipuncture into SST tubes pre-dose, Day 1 (immediately after the end of the infusion and 4 hours after the end of the infusion), and Days 3, 8, 15, 22, 29, 43, 57, 71, and 85. For Group 1 animals, blood (1.5 mL each) was collected by venipuncture into SST tubes pre-dose, Day 1 (immediately after the end of the infusion and 4 hours after the end of the infusion), and Days 3, 8, 15, 22, 29, 43 (predose and 4 hours after the end of the infusion), 50 (predose and 4 hours after the end of the infusion), 57 (predose and 4 hours after the end of the infusion), 59, 64, 71, 78, 92, 106 and 120. Samples were analyzed for pharmacokinetic evaluation and for serum M-CSF activity and remaining heRX1-10.G1 activity.

Blood samples were allowed to clot at room temperature for approximately 30 minutes prior to centrifugation. The serum was obtained by centrifugation at approximately 2700 rpm for 10 minutes at approximately 4° C. and the resultant serum were divided into 4 aliquots.

All of the above U.S. patents, U.S. patent application publications, U.S. patent applications, foreign patents, foreign patent applications and non patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

We claim:

1. A method of treating a patient suffering from an osteolytic disorder comprising the steps of administering to said patient an anti-M-CSF antibody at a monotherapeutically effective dose and an osteoclast inhibitor at a monotherapeutically effective dose.

2. A method of treating a patient suffering from an osteolytic disorder comprising the steps of administering to said patient an anti-M-CSF antibody at a monotherapeutically effective dose and a bisphosphonate at a monotherapeutically effective dose.

3. The method of claim 1 wherein the osteoclast inhibitor is a RANKL inhibitor.

4. The method of claim 2 wherein the anti-MCSF antibody is RX1-derived antibody, an MC1-derived antibody, an MC3 derived antibody, or 5H4-derived antibody.

5. The method of claim 2 wherein the anti-MCSF antibody is heRX1.

6. The method of any of the preceding claims wherein the osteolytic disorder is osteoporosis, bone loss associated with cancer metastasis, Padget's disease, or periprosthetic bone loss.

7. The method of any of the preceding claims wherein the osteoclast inhibitor and the MCSF antibody are administered simultaneously

8. The method of claim wherein the RANKL inhibitor is selected from the group consisting of an anti-RANKL antibody, a soluble RANKL receptor, and a RANKL vaccine.

9. The method of claims 3, 4, 6, and 7 wherein the anti-RANK antibody is AMG-162.

10. The method of claims 2, 4, 6 and 7 wherein the bisphosphonate is selected from the group consisting of is zoledronate, pamidronate, clodronate, etidronate, tiludronate, alendronate, ibandronate, and risedronate.

11. The method of claim 10 wherein the bisphosphonate is zoledronate.

12. A method of treating a patient suffering from an osteolytic disorder comprising the steps of administering to said patient an anti-M-CSF antibody at a monotherapeutically-effective dose and a bisphosphonate at a monotherapeutically effective dose for a transition period.

13. The method of claim 12 wherein the osteoclast inhibitor is a bisphosphonate or a RANKL inhibitor.

14. The method of claim 13 wherein the transition period is approximately 1-7 days.

15. The method of claim 13 wherein the transition period is 1 week to 1 month.

16. The method of claim 13 wherein the transition period is 1 month to 3 months.

17. The method of claim 13 wherein the transition period is 3 to 6 months.

18. The method of claim 13 wherein the transition period is 6 to 12 months.

19. The method of claim 13 further comprising the step of discontinuing the bisphosphonate therapy after the transition period.

20. The method of claim 13 further comprising the step of reducing the dose of bisphosphonate after the transition period.

21. The method of claim 13 wherein the dose of bisphosphonate is reduced immediately after the transition period.

22. The method of claim 13 further comprising the step of reducing the dose of anti-MCSF antibody after the transition period.

24. The method of claim 13 wherein the dose of anti-MCSF antibody is reduced immediately after the transition period.

25. The method of claim 13 wherein the bisphosphonate is administered at least one-time after the transition period.

26. The method of any of the preceding claims wherein the anti-MCSF antibody comprises at least two of the CDRs from murine RX1 antibody of SEQ ID NO: 5 and SEQ ID NO: 6.

27. The method of any of the preceding claims wherein the anti-MCSF antibody-comprises at least three of the CDRs from murine RX1 antibody of SEQ ID NO: 5 and SEQ ID NO: 6.

28. The method of any of the preceding claims wherein the anti-MCSF antibody competes with murine RX1 antibody for binding to M-CSF of SEQ ID NO: 5 and SEQ ID NO: 6 by at least 75%.

29. A method of claims 6, 13, and 20, wherein the osteoclast inhibitor is zoledronate and the MCSF antibody is an RX1-derived antibody.

30. The method of claim 29 wherein the zoledrorate is administered at a dose of between 0.5 mg and 4 mg.

31. The method according to claims 4, 6, 8, 10, 11, or 12 wherein the anti-MCSF antibody binds to the same epitope as RX1, MC1, MC3, or 5B4 antibodies.
